(19) United States
(12) Patent Application Publication
(10) Pub. No.: US 2023/0041901 A1
(43) Pub. Date: Feb. 9, 2023

(54) READILY INTERCHANGEABLE LIGHT MODIFIER FOR A UV C FIXTURE

(71) Applicant: Lumenlabs LLC, Poway, CA (US)

(72) Inventors: Kevin C. Baxter, Tulsa, OK (US); Scott R. Gant, Poway, CA (US)

(21) Appl. No.: 17/964,796

(22) Filed: Oct. 12, 2022

Related U.S. Application Data
(63) Continuation-in-part of application No. 17/327,499, filed on May 21, 2021.
Application No. 17/327,499 is a continuation of application No. 17/156,426, filed on Jan. 22, 2021, now Pat. No. 11,357,879.
Application No. 17/156,426 is a continuation of application No. 17/080,390, filed on Oct. 26, 2020.
(60) Provisional application No. 63/346,584, filed on May 27, 2022.
(60) Provisional application No. 63/328,491, filed on Apr. 7, 2022.
(60) Provisional application No. 63/313,481, filed on Feb. 24, 2022.
(60) Provisional application No. 63/254,843, filed on Oct. 12, 2021.

(51) Int. Cl. A61L 2/24; G01C 3/00; A61L 2/10; G01J 1/42; A61L 2/26; H01J 61/02; H01J 61/12; H01J 61/40; H01J 61/52; H05B 41/38

(52) U.S. Cl. CPC A61L 2/24; G01C 3/00; A61L 2/10; G01J 1/42; A61L 2/26; H01J 61/025; H01J 61/12; H01J 61/40; H01J 61/52; H05B 41/38; A61L 2202/14; A61L 2202/11; A61L 2202/25

(57) ABSTRACT

A UV C light source including a UV C bulb adapted to emit and project UV C light at a wavelength and an interchangeable UV C light modifier through which at least a portion of the UV C light emitted from said UV C bulb is projected. The UV C light modifier may be reflective, such as a reflector, perforated, holographic material, or mechanical modifier such as a barn door. The UV C light modifier might produce a narrow pattern, circular pattern, flat pattern, or asymmetrical pattern or other desired geometric pattern, or upper air pattern. The UV C light modifier is easily removed and interchanged or may be selectable such as by receiving a base UV C fixture including the UV C light source of the present invention and selecting a desired light modifier.

### CROSS-REFERENCE TO RELATED CASES

This application claims the benefit of U.S. provisional patent application Ser. No. 63/346,584, filed on May 27, 2022, and U.S. provisional application 63/328,491 filed on Apr. 7, 2022, and U.S. provisional patent application Ser. No. 63/313,481 filed Feb. 24, 2022, and U.S. provisional patent application Ser. No. 63/254,843, filed on Oct. 12, 2021, and is a continuation-in-part of U.S. patent application Ser. No. 17/327,499 filed on May 21, 2021 which claims the benefit of U.S. provisional patent application Ser. No. 63/183,937 filed on May 4, 2021 and is a continuation-in-part of U.S. patent application Ser. No. 17/459,285 filed on Aug. 27, 2021 which is a continuation of U.S. patent application Ser. No. 17/193,839 filed on Mar. 5, 2021 which is a continuation of U.S. patent application Ser. No. 17/156,426 filed on Jan. 22, 2021 which is a continuation of U.S. patent application Ser. No. 17/080,390 filed on Oct. 26, 2020 which claims the benefit of U.S. provisional patent application Ser. No. 63/069,436 filed on Aug. 24, 2020 and incorporates such applications by reference into this disclosure as if fully set out at this point.

### FIELD OF THE INVENTION

The inventive system is in the field of Ultraviolet Light sterilization, specifically in the C band of wavelengths (UV-C). Such sterilization is presently used in hospital surgery rooms, burn wards, and similar areas that require a high degree of sterilization. The primary difference with these existing uses is the inventive system will be used safely in the presence of people and living tissues.

### BACKGROUND OF THE INVENTION

UV-C is recognized as one of the most effective wavelengths at killing the small pathogens because the shorter the wavelength the more powerful it is. Only recently was it discovered that some of the wavelengths in this band are long enough to kill pathogens and short enough to not be able to penetrate living cells. Living cells are many times larger the tiny pathogens that we want to kill. UV-C is from 100 nm to 280 m, and the wavelengths that are generally being considered safe for exposure to human tissue are from 200 nm to 230 nm. UV-C does generate undesirable ozone, especially at wave lengths shorter than 200 nm.

Several studies have shown that hairless mice can be subjected to over 20 times the amount of 200 nm to 230 nm UV C as is presently suggested for humans, 8 hours a day, with no adverse effect. These studies have been performed in Japan at University and in the US at Columbia University. These studies are extending in time for up to 6 months, still with no adverse effects. Recently humans in Japan were also tested to 250 times the exposure of what is needed to kill 99.9/6 of pathogens and the test subjects showed no adverse effects, no sunburns, nothing.

There are several technologies that can generate UV light in the germicidal wavelengths, gas-discharge lamps have been around a long time and depending on the gases used can kill pathogens. Low pressure mercury generates 254 nm and has been the standard for decades, it is basically a fluorescent light without the phosphors on the inside that convert the UV to visible light. LEDs have recently been commercialized in the UV-A and UV-B spectrums, but they are inefficient. There are a few in the longer wavelengths of the UV C spectrum. A research project in Japan recently made an LED that was near 225 nm, the safer portion of the UV-C spectrum, but it was very inefficient but nevertheless is being commercialized now. There are several of these chemistry/proportion generated LED technologies using aluminum gallium nitride and/or magnesium zinc oxide that will need some filtering if it can generate enough light to sanitize. There are LED bandgap technologies such as NS Nanotech's nitride semiconductor, where the wavelength is determined by the physical size of the light emitting elements, that can generate light in this same spectral region, and they might not need filtration because bandgap LEDs will generate a nearly single wavelength output.

Several companies are making UV-C excimer fixtures that emit 222 nm such as Ushio's 12 W Care222, and another by Eden Park's Flat Excimer Lamps. The Ushio fixture has a flat faceplate filter that is separate from the bulb that blocks all unwanted spectrum that the bulb generates, and that spectrum is any wavelength longer than 230 nm when measured at the incident angle of zero or 234 nm for 1% of peak or 237 nm for off angle light that is completely attenuated. As the angle of light to the filter becomes greater the light's path through the filter and glass becomes longer, filter layers appear thicker to light on this path allowing wavelengths that are longer than the cutoff to pass, though very attenuated. Hence the difference between cutting off at 230 nm at a zero incident angle and passing some small attenuated light at angles, down to almost 237 nm. The 7 nm difference is about the limit before the angle becomes so great that all light is attenuated, a 4 nm difference passes 1% of peak. This is an unwanted feature of filter technology but one that has to be recognized. Also the quartz glass and hafnium by themselves cause attenuation at 50 nm and 130 nm intrinsically. These are unwanted attenuations but have no impact on the desired wavelengths because no light generated is near those attenuated wavelengths.

The Eden Park device has no filter attached at this time and consequently emits 25% of its energy in the dangerous wavelengths from 239 nm to at least 380 nm. Luckily these bulbs are not very bright and are not strong enough to do too much harm.

When these filters are not in place then these lights will emit spectrum that is not safe for living tissue. If a maintenance worker were to try and replace a bulb they could be exposed to harmful light. If the glass filter broke or degraded the user would be in danger.

Lastly the materials used are critical. UV-C cannot penetrate plastics and many glasses, only quartz glass can be used without huge losses or downright failure to emit the UV-C light. Even nitrogen and moisture in the air will also absorb or block the UV-C if it is transmitted too far through the air.

What is needed is an affordable and efficient UV sterilization light that would be good at killing pathogens with no chance to harm, under any situation, humans that would be present.

### SUMMARY OF THE INVENTION

The inventive device provides a human-safe UV-C sterilizing fixture that can be used in continuous public places. The fixture will be safe in all situations, efficient, affordable, and could monitor itself and report conditions. The present disclosure includes, generally, a UV C light source including a UV C bulb adapted to emit and project UV C light at a wavelength and an interchangeable UV C light modifier through which at least a portion of the UV C light emitted from said UV C bulb is projected. The UV C light modifier may be reflective, such as a reflector (mirror, PTFE/Teflon), perforated, holographic material (such as evaporative plating or chrome to provide a very predictable pattern), or mechanical modifier such as a barn door. The UV C light modifier might produce a narrow pattern, circular pattern, flat pattern, or asymmetrical pattern or other desired geometric pattern, or upper air pattern. The UV C light modifier is easily removed and interchanged or may be selectable such as by receiving a base UV C fixture including the UV C light source of the present invention and selecting a desired light modifier (all considered “interchangeable” as used herein). In this way, the fixture, as well as the selected interchangeable light modifiers could be considered a kit.

Studies at Columbia University show that pass filters tuned from 200 nm-230 nm, measured at an angle of 0 degrees incident angle (perfectly perpendicular to the filter plane), kills the pathogens and don't hurt human cells and this is shown in prior art devices. Recent testing and observations by the inventors show that truly dangerous wavelengths start between 239 nm and 240 nm. And studying the energy curve just below 230 nm shows a very low energy being emitted by the bulb for the next several nanometers and human cells are not penetrated until about 240 nm. This would allow changing the cutoff filter in the inventive device from 230 nm to 231 nm, or 232 nm, or 234 nm, or 235 nm, 236 nm, or even as high as 237 nm before harmful wavelengths have sufficient energy, above 1% of the 222 nm peak, for the need to be filtered which is at 240 nm. The inventive device would use 207 nm or 222 nm excimer technology or LEDs combined with an integral band pass filter that would block spectrum with wavelengths longer than 236 nm and intensities of greater than 1% of the emitter's peak wavelength. When describing this type of filtering technology it is important to understand that the filtering is never absolute. The filtering can reduce intensity by a few percent to more than 90% and when one attempts to filter a particular wavelength it is partial, with filtering to adjacent wavelength to a lesser degree, and the second adjacent wavelengths even less. There is a 3 or 4 nanometer slop to the technology. That is why the inventive device begins it filtration at 236 nm in order to be sure to block 240 nm emissions.

A small change to the filtration by beginning the starting point of 234 nm instead of 230 nm allows 2.5 times more usable emitted energy than the 200-230 nm filters, there is higher transmission and wider transmission of wavelengths in the safe region. The filter material could be deposited directly on the bulb's envelope, which would be made of quartz glass, and this would block all harmful light even when handled during installation or maintenance. There are several well understood spikes of dangerous wavelengths in both the 207 nm and 222 nm bulbs, the evolving LED solutions are still changing so it is hard to be absolute regarding the filtering requirements of the LEDs until their commercialization is complete and fixed, but there will be a couple of undesirable spikes.

Ideally the inventive dynamic 236 nm filter would avoid having useless filtering for areas of the bulb's spectrum where little or no energy is generated in the first place, and then complete filtering in the high energy or dangerous portions. The dynamic filter would not need to filter shorter wavelengths than 236 mm because the 222 nm bulb does not emit harmful levels of light above 200 nm.

The 222 nm bulb also does not generate energy in the 262 nm to 288 nm range that are greater than 1% of the 222 nm peak intensity. Not having filters in this area also would allow for less filter layers. There would ideally be additional filtering in the upper portion of the UV B spectrum, specifically from 288 nm to 315 nm as these wavelengths are in the dangerous zone and would be generated above 1% of the 222 nm peak by the excimer bulb or LED. Wavelengths longer than 315 nm are generally considered safe at even high exposures. These inventive ideas combined could reduce the number of required layers from the traditional 80 plus layers down to less than 60 layers for a dynamically filtered design that also has higher overall transmission and a lower cost. Certainly the number of layers could drop to under 75 with very little optimizing.

Partial filtering is another dynamic way that could also be applied to minimize opacity and losses by filtering a couple of wavelengths, and then skipping the next few wavelengths, and then filtering the next couple of wavelengths, and then skipping the next few wavelengths, and so on. There is partial blocking of the adjacent skipped wavelengths for at least 3 or 4 nanometers so there are not big spikes of dangerous light getting through the skipped portions. This technique could be used in areas where the dangerous wavelengths are a few % of the 222 nm peak intensity, or are at the edges of the dangerous band of wavelengths. Varying the number of wavelengths filtered to the number of wavelengths skipped sequences could adjust the filtering percentage from high to low or from low to high depending on the need to reduce a wavelength's intensity to below 1% of the 222 nm peak intensity, but not wastefully lower.

Because these types of filters form a narrow output beam it would be desirable to widen or shape the beams. Diffusion or other light modifiers such as lenses and or reflectors could be added after the filter to widen or change the light beam's shape. Studies in Scotland showed that a low level irradiation using Far UV C over a wide beam or area is much more effective at killing the first 99% of virus and bacteria than a powerful but narrow beam in the same environment. Ideally these light modifying elements would be able to snap on to the fixture for quick changes in the field or while installing the fixture to best optimize it for the unique geometry of the space that it is being installed in.

The 207 nm version requires the gasses bromine (Br) and argon and krypton (Kr). The 222 nm version uses krypton (Kr) and chloride (Cl). The filter material would ideally be very pure hafnium oxide deposited 2˜3 um building a cutoff filter 236-315 nm with a depth of approximately 0.0001. Aluminum oxide can also be used but because of the number of layers that are required makes it is a more expensive process than that using hafnium, but a better efficiency could be achieved with using both the hafnium and the aluminum oxide. This type of excimer bulb ideally uses Dielectric Barrier Discharge (DBD) and this is where the two primary electrodes are on the outside of the quartz envelope and in order to get the gasses to excite requires very high voltages, in the thousands of volts. Consequently, the gasses inside of the envelope will not be in contact with any metals that could contaminate them. Some Ushio lights have one conductor inside the envelope, sort of a hybrid, short-arc/DBD bulb. The inventive device avoids the problems of dissimilar materials and envelope contamination, and multiple types of glass needed, by keeping all of the electrodes on the outside of the envelope. This inventive bulb does need a higher arc voltage but that is a small challenge for all of the benefits that a quartz only envelope solution provides.

The integral band pass filter could be deposited on a separate piece of quartz that would be permanently attached to the bulb's envelope using UV compatible adhesive around the edges. The filter would be integral to the finished assembly. This would protect users from UV exposure under all conditions including bulb changing and maintenance. Sealing around the perimeter would also allow for an inert gas to fill around the bulb, lowering the amount of ozone generated. The inert gas could be nitrogen or one of the noble gasses, or any gas that doesn't block or react with the light generated.

The inventive safe DBD device would also have an integrated captured reflector on the backside of the bulb's envelope and an additional 2 integral mirrors, one on each side of the bulb, set at 45 degrees to the filter face in order to maximize the light out. Light passing through the filter is heavily attenuated if it passes through at any angle other than absolutely perpendicular to the filter's plane. Light passing through at even 10 degrees from perpendicular is attenuated by over 50%, depending on the filter's composition, and the greater the angle the greater the attenuation and absorption. In order to reduce the cost of the bulb the filter could be separate because the filter is the most expensive part of the bulb. Disposing of such a costly component each time a bulb is replaced raises the total cost of ownership of the fixture. A clear faceplate could be added in its place on the bulb cartridge and the filter would be mounted permanently on the fixture. This could allow several filters to be used for each bulb, each filter positioned such that they all were tilted exactly perpendicular to the paths of light generated by a bulb, thus raising the overall efficiency of the fixture.

Ideally the reflector could be a separate material that would be permanently connected to the bulb's envelope using UV compatible adhesive around the edges. The shape of the bulb would ideally be a flattened quartz tubing where the two flattened sides are parallel to each other. This design would be the cheapest and easiest configuration and is scalable. Other desirable but less optimum bulb shapes could be a tube-based design that has a complex shape which would allow the reflector on the back side to be optimized for different beam angles and patterns. A flattened circular tube-based bulb would emit a Lambertian pattern if desired.

The inventive safe DBD bulb will be cartridge based as to be easy to replace with rigid exposed conductors. The lifetime of 222 nm excimer bulbs is generally about 8000 hours, after they lose 10% brightness it goes down quickly from there. The high voltages and drivers of excimer bulbs are unique and cannot be connected to other power sources so each different bulb power/size will need a unique connector to avoid connections between electrically incompatible parts and polarization would be desirable as well. The primary high voltage electrode would ideally be a conductive ink made of silver or similar conductive metals printed in a mesh pattern over the non-opaque or filter area. The second primary electrode would also be at o volts and use the conductive ink as this would reduce parts. Because of the high voltages used in excimer bulbs a safety cutoff switch should be included in the inventive fixture in the case of a maintenance worker opening the fixture to safely replace a bulb.

Such a safe bulb will also have a built-in smart chip that has non-resettable serial number, manufacture date, use date, temperature boundary, and a Hobbs meter or hour meter. The smart chip would ideally use encryption in order to not be hacked. The bulb's fixture will be in communication with the smart chip on the bulb, and the fixture will have Internet Of Things (IOT) connectivity. The bulb's fixture will monitor the bulb life and when it was first turned on and shut down the bulb, if it or is it running over temperature, if it is near to end of life?

This will protect the users in case the filters begin to degrade, or the light intensity is not up to specification. It can notify or be polled by maintenance software and request replacement. The IoT connection can be used to talk to remote sensors that measure the output at different locations around the fixture's environment, where people are exposed. The IoT could also report temperature, air particles, proximity, human movement, or any other sensor in the fixture. The IoT could control the output of the light including power levels, timing, schedules, etc. In one particular embodiment a fixture of the present disclosure may be an IoT fixture (and may include an IoT interface board) mounted on or near a ceiling (or wall etc.) in an occupiable space and may be one of a plurality of fixtures wherein one or more is an IoT fixture. It is contemplated in a method that Threshold Limit Value (“TLV”) exposure may be adjusted using a hand held irradiance meter (light meter) communicating via IoT to the fixture or another particular fixture of a plurality of fixtures using IoT. The fixture could then adjust its brightness regardless of the reflectivity of the ceiling to proper TLV limits based on the meter's reading.

Because this type of bulb does not emit much visible light the fixture should include a multicolored LED indicator or LCD so that users can quickly at a glance know that the fixture is working, or not working properly. The bulb would also have a break wire running through the body and external glass so that if the body or glass were ever broken the break wire would also break and cause a bulb shutdown.

Because the light output degrades over time and the inventive safe fixture has feedback as to the environment's light level the fixture could boost the output over time to have a constant output level. The fixture would ideally have a light sensor to determine light output. The output level could be estimated by time used and that table could be programmed into the fixture so that the fixture could be constantly increasing the output power for a near-constant lumen output, or at least a good estimation.

When emitting a light that kills germs and the public is exposed to it, absolute safety is the primary standard that has to be met, tested and verified. The safeguards built into this fixture should not be luxuries, but should be requirements to allow a UV-C light to be exposed to the public.

When entering an area that is protected by the inventive device, the public should have access to the assembled data. They might ask, “How long is the kill time for pathogens on surfaces, in the air? At what percentage output is the system running at? How much time can a human spend in this environment per day?”

The nature of DBD excimer bulbs is that the gasses can overheat and that causes the lighting level and lifetime to diminish so proper cooling is a requirement. The inventive safe bulb could have a ceramic or metallic heatsink on the back side. The envelope could also be extruded with linear fins to add surface area for convection cooling, similar to how an aluminum heat sink is designed. The fins would be on the outside of the light emitting envelope. A fan could blow air on the bulb in order to lower the gas temperature, especially when the power level is raised. A temperature sensor could part of the bulb to give temperature feedback and the fan's speed could be regulated as to have a constant envelope temperature. The fan could be powerful enough to slightly stir the air in the area or room that it is supposed to sanitize, especially if the air was very stagnant. This would bring airborne pathogens closer to the sanitizing rays of light that might otherwise be shaded or too far away to be killed.

Because the high voltage of excimer technology can generate small amounts of ozone the inventive fixture would have a 265 nm LEDs in a shrouded and highly optically reflective air exhaust chamber. This way the small inefficient 265 nm LEDs energy could eradiate the ozone while bouncing around the reflective chamber and remove ozone from the air exhaust before it leaves the fixture.

Another reality of excimer bulbs is that they sometimes are hard to start in cold conditions and they need coiled filaments, resistors, or similar heating elements to preheat the gasses. These could be used to preheat the safe UV-C bulb at cold or even at normal start-ups and could be either inside or ideally outside the quartz envelope or encased in the ceramic heatsink. The power supply in the fixture would have to have additional circuitry to enable this feature. The inventive fixture would use pulse square wave rather than sine wave to drive the bulbs. Sine wave power for these high voltage applications are the standard but all of the energy below the peak voltage of the sine wave does not convert to light, it only makes heat.

The inventive safe device can use either 222 nm or 207 nm chemistries or both. Using two separate envelopes would allow tailoring the specific wavelength to best kill an emerging pathogen. Each chemistry has different drive voltages and arc gaps, but a power supply could easily be configured to drive either or both simultaneously. Ideally the inventive device will use DBD where the electrodes are on the outside of the glass envelope. This type of discharge requires very high voltages to get the gasses inside to excite but using this technique the gasses inside the envelope are never contaminated by electrode erosion, a common problem in gas discharge lamps when used over time. It would be desirable to combine the 222 nm and 207 nm gasses in a single bulb for greater efficiencies. The bromide and chlorine are in the same chemical family and could add to each other's strengths.

The inventive safe bulb would be used in environments where there is regular visible light coming from light fixtures and the inventive bulb could be combined with traditional light sources in a single fixture. If there was any adverse visible color emitting from the UV-C portion of the fixture the visible light's spectrum could be modified and mixed in such a way as to normalize the mixture or average of color coming from the fixture. This type of fixture would ideally be a “can”, the type of fixture that is installed in a round or square hole in a ceiling.

It would be possible to use a 222 nm bulb without a filter or a UV LED in an occupiable space if the ultraviolet energy were directed upwards and outwards and not below 2.1 meters from the floor. This is called upper air disinfection. Approximately half of all air in an occupied space is above the heads of the room's occupants. Light modifiers such as lenses, diffusion, and reflectors could shape the light to evenly cover a room without radiating in the direction of the floor.

The inventive safe system could be packaged as a typical light bulb. The ballast or power supply could be fitted in the base and the bulb would shine omnidirectionally, just like an LED or compact fluorescent light bulb, and it could have conventional lighting included as well.

### BRIEF DESCRIPTION OF THE DRAWINGS

FIG. 1 Low pressure mercury bulb (prior art) line drawing from photo.

FIG. 2 Ushio Care222 excimer bulb and driver (prior art) line drawing from photo.

FIG. 3 Eden Park's Flat Excimer Lamp (prior art) line drawing from photo.

FIG. 4 Spectrum graph of a 207 nm Br Kr emission.

FIG. 5 Spectrum graph of a 222 nm Kr Cl emission.

FIG. 6 Spectrum graph of a 207 nm Br Kr emission with proper filtration.

FIG. 7 Spectrum graph of a 222 nm Kr Cl emission with proper filtration.

FIG. 8 Drawing of basic bulb.

FIG. 9 Exploded isometric drawing of bulb assembly.

FIG. 10 Side view section of bulb assembly.

FIG. 11 Isometric view of bulb cartridge.

FIG. 12 Exploded view of multi-bulb fixture.

FIG. 13 Depiction of fixture aiming down with bulbs aiming straight down.

FIG. 14 Depiction of fixture aiming down with bulbs aiming out at 45 degrees.

FIG. 15 Safe bulb heat sink with heating filament.

FIG. 16 Safe Bulb with both UV-C and general illumination elements.

FIG. 17 Temperature regulated fixture with fan-cooled safe bulb.

FIG. 18 Block diagram of a fixture including driver, bulb, temperature sensor, and safety cutoff switch in a fixture.

FIG. 19 Network diagram of IoT bulb and system.

FIG. 20 Depiction of the output of a pulse wave power supply compared to a sine wave.

FIG. 21 Depiction of the output of a pulse wave power supply at full power and during dimming.

FIG. 22 Depiction of a focusable UVC light for wider beam angles.

FIG. 23 Depiction of a single-phase dielectric fluid cooled UV C bulb.

FIG. 24 Depiction of a single-phase dielectric fluid cooled UV C system.

FIG. 25 Depiction of a low voltage UVC bulb system.

FIG. 26 Depiction of a low voltage inductively powered UV C bulb system.

FIG. 27 Isometric view of a safety break wire in a bulb cartridge.

FIG. 28 Depiction of a schematic of a safety break wire system.

FIG. 29 Simplified view of single filter bulb (prior art)

FIG. 30 Simplified view of single filter bulb with reflector (prior art)

FIG. 31 Side view of inventive device with 2 filters

FIG. 32 Side view of inventive device with 3 filters

FIG. 33 Side view of inventive device with 4 filters

FIG. 34 Side view of inventive device with cylindrical filter

FIG. 35 Side view of inventive device with arc filter

FIG. 36 View of inventive device with spherical filter

FIG. 37 Side view of cylindrical filter being fabricated in a reactor

FIG. 38 Isometric view of a multifaceted omnidirectional UV C tower

FIG. 39 Side view of a room being sanitized by the safe UV C fixture with a powerful fan

FIG. 40 Isometric view of a simple cartridge bulb

FIG. 41 Exploded view of a simple cartridge bulb and filter assembly

FIG. 42 Isometric view of a simple cartridge bulb and filter assembly

FIG. 43 Exploded view of a single bulb fixture that uses multifaceted filters

FIG. 44 Isometric view of a single bulb fixture that uses multifaceted filters

FIG. 45 Cross section view of a directional high intensity filtered UV source

FIG. 46 Isometric view of a directional UV C tower

FIG. 47 Isometric view of a directional UV C gateway tower

FIG. 48 Side view of two directional towers forming a UV C gateway

FIG. 49 Logarithmic graph of an unfiltered 222 nm

FIG. 50 Graph of relative spectral effectiveness

FIG. 51 Graph of filter's pass curves

FIG. 52 Isometric view of a ozone reducing chamber

### DETAILED DESCRIPTION OF THE PREFERRED EMBODIMENTS

Before explaining the present invention in detail, it is important to understand that the invention is not limited in its application to the details of the construction illustrated and the steps described herein. The invention is capable of other embodiments and of being practiced or carried out in a variety of ways. It is to be understood that the phraseology and terminology employed herein is for the purpose of description and not of limitation.

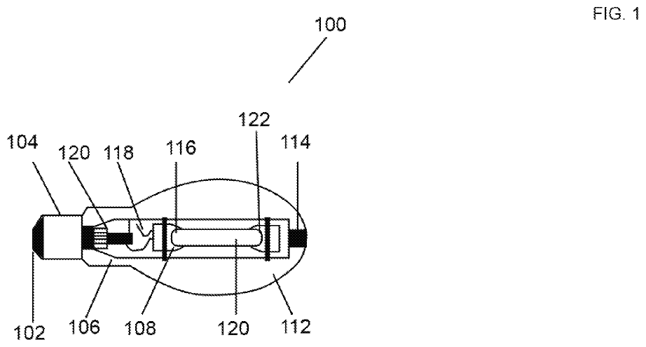

Referring now to the drawings, wherein like reference numerals indicate the same parts throughout the several views, a representative depiction of an (existing art) Low pressure mercury bulb 100 shown in FIG. 1. Where bulb 100 has several parts, the electrical contact points 102 and 104, the metal support structure 106, the primary electrode 108, the mercury and argon gas 110, the outer envelope 112, tip support 114, the striking electrode 116, a resistor 118, a glass base 120, and the second primary electrode 122.

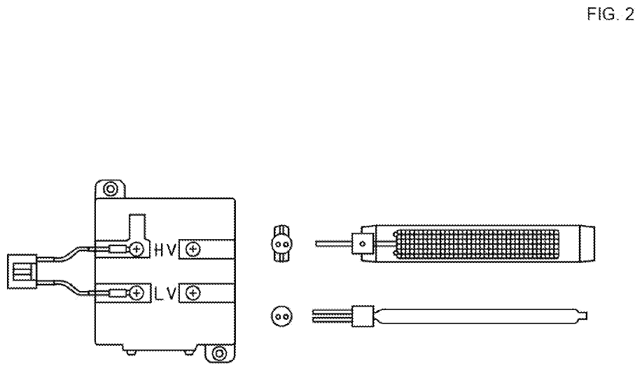

A newer and different technology is shown in FIG. 2 where an Ushio Care222 excimer bulb and driver system 200 is shown in block diagram form. The bulb 202 is driven by the driver 204 through 2 wires 206 and 208. The bulb has two primary electrodes 210 and 212. Housings and filters are separate and not shown.

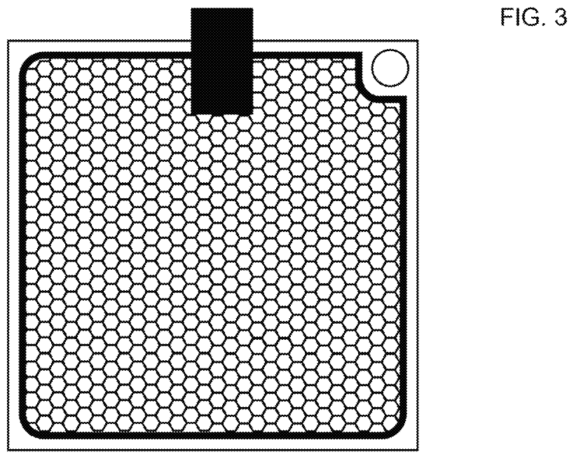

The next drawing in FIG. 3 shows a flat version of an excimer bulb that is Eden Park's Flat Excimer Lamp 300. It has two narrowly spaced flat sheets of quartz glass 302 and 304 with electrodes 306 and 308 on the outside of the front 310 and back 312 plates. The bulb has no filter or reflector.

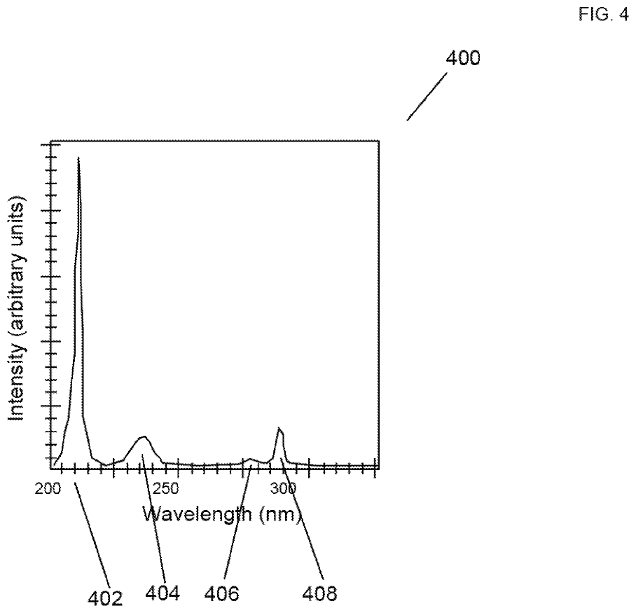

In FIG. 4 a spectral graph is shown of a 207 nm Br Kr emission 400. The primary emission spike 402 at 207 nm is a safe wavelength for human tissues and is deadly to small pathogens such as viruses and bacteria. Also shown is a small amount of emission at 230 nm 404 which is just at dangerous edge of spectrum incompatible with human exposure. Shown is additional emission at 270 nm 406 which even at this low level is extremely dangerous to human exposure. Lastly a spike of emission at 290 nm 408 and it is not safe for human exposure.

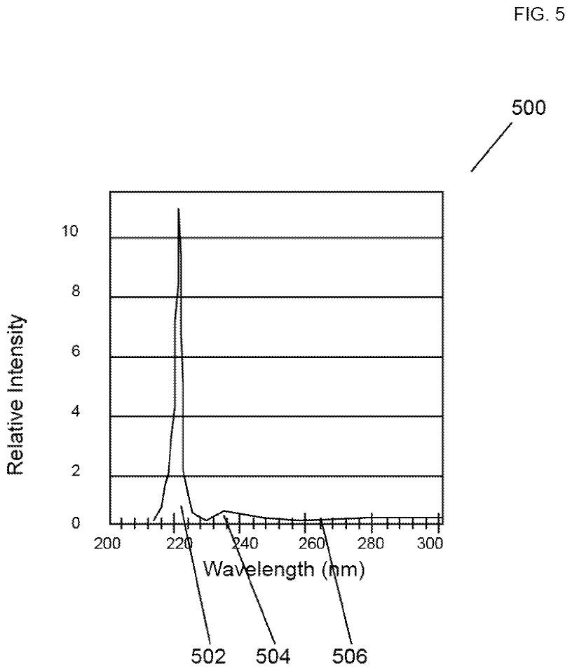

In FIG. 5 there is a spectral graph of a different chemistry than what was shown before, this 222 nm Kr Cl emission 500. The primary emission spike 402 at 222 nm is a safe wavelength for human tissues and is deadly to small pathogens such as viruses and bacteria but energy curve 500 continues at a low level but rises into the deadly 238 nm 504 range. There is also a small bump of emission at the 258 nm range 506 and this is extremely dangerous even at such a low level. Note the flat low areas in between these peaks that would require virtually no filtering to be safe.

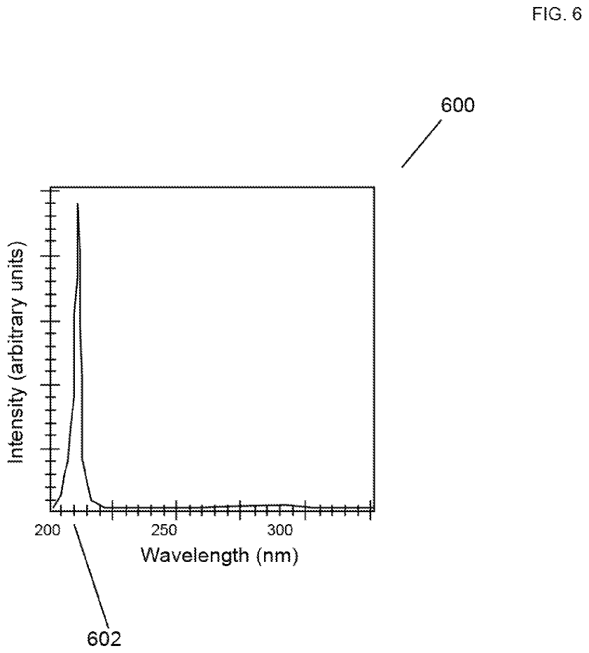

In FIG. 6 a spectral graph is shown of a safe 207 nm Br Kr emission 600 with a spike at 207 nm 602. 207 nm is a safe wavelength for human tissues and is deadly to small pathogens such as viruses and bacteria and due to subtractive filtering, there is no emissions in the dangerous wavelengths.

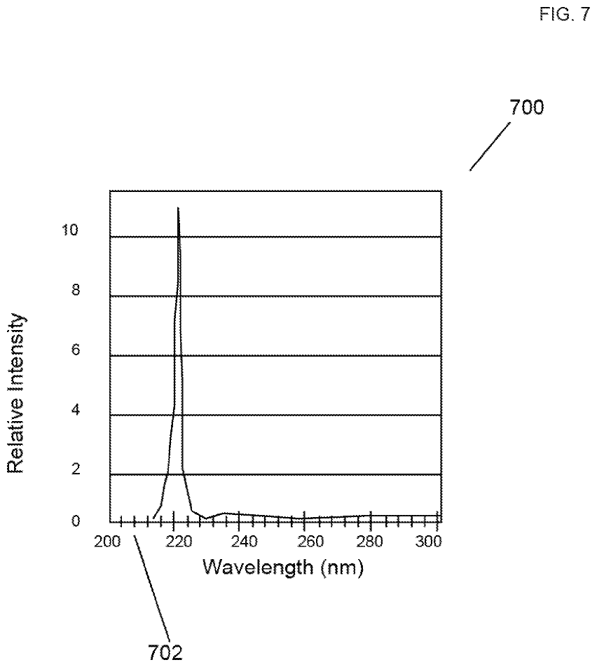

In FIG. 7 a spectral graph is shown of a safe 222 nm Kr Cl emission 700 with a spike at 222 nm 702. 222 nm is a safe wavelength for human tissues and is deadly to small pathogens such as viruses and bacteria and due to subtractive filtering, there is no emissions in the dangerous wavelengths.

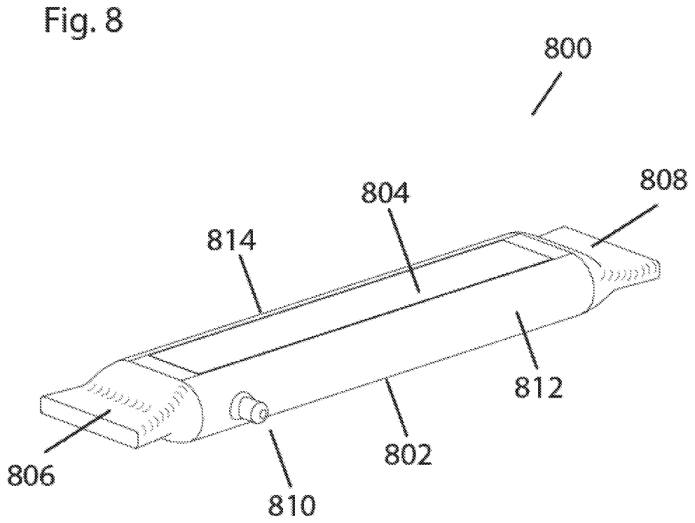

In FIG. 8 an inventive excimer bulb 800 is shown. The bulb 800 has a quartz envelope 802 that contains a combination of gases at about 300 millibar pressure, depending on the specific chemistry involved, Br Kr at 207 nm or Kr Cl at 222 nm. The two flattened sides are parallel to each other and are approximately 10 mm apart, but this varies with different power levels, fill pressures, and drive voltages.

The quartz envelope 800 starts as a round cylinder and is heated and pulled through rollers that flatten the two sides, the front face 802, and the back face 804 to be parallel with each other. The ends of the flattened tube are then sealed at both ends 806 and 808 by heat welding to seal them. The fill point 810 as shown starts as a small fill tube that is melted shut after the bulb has been cleaned and filled with the low-pressure gasses. The sides of the bulb 812, and 814 allow light to pass as well, the right side 812 and the left side 814. These pathways of light have been ignored by prior art devices and enormous amount of wasted optical energy will be harnessed here by the inventive device.

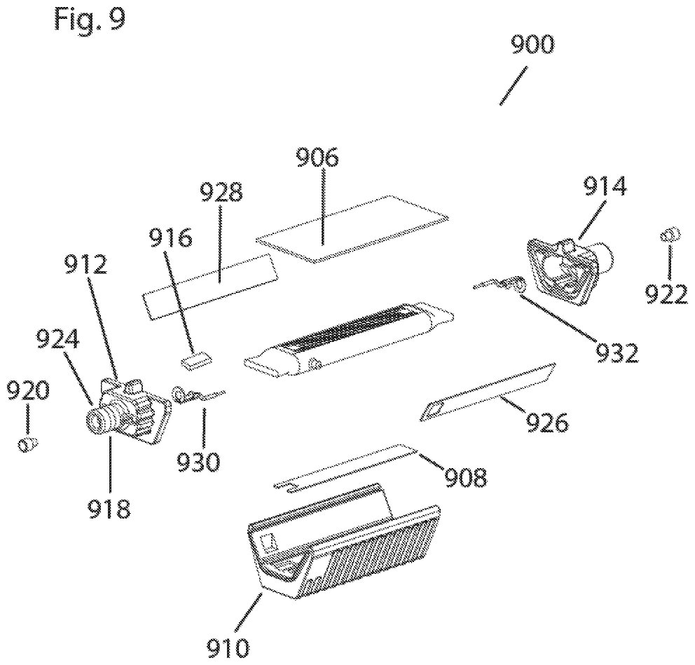

In FIG. 9 depicts an exploded view of a safe flattened tube design bulb assembly cartridge 900. Starting at the bulb 800 as the center of the assembly. The front electrode 902 is the ground, or 0 Volt, electrical grid and it is placed directly against the quartz bulb's face 802. It can be made of a non-corroding conductive metal such as molybdenum or silver. Ideally this front grid 902 or screen will be closest to the user. To eliminate ozone production the grid would be applied as a conductive liquid or ink to eliminate oxygen from the electrical path. Similarly, the rear electrode 904 is the positive electrode. It too is placed directly against the quart envelope's back side 804. The voltage on these sides are approximately 4,000 10,000 Volts AC and are placed away from the user as possible. The back electrode 904 would be plated on or applied as a conductive ink to eliminate oxygen from getting between the conductive electrode and the path of high voltage electricity. The bulb could ideally be coated with evaporated quartz glass to protect the electrodes from oxygen. This is similar to the filter making process and would be very quick and inexpensive to implement.

A rear reflector 908 is generally flat but could be curved and is added against the outside of the rear electrode 904. The rear reflector 908 and the rear grid 904 could be combined as one part to both conduct electricity and reflect light, and as such could be a vapor plated on aluminum layer which would be deposited directly to the back side face 804 of the bulb 800 to further minimize parts and costs. Side reflectors 926, and 928 are set at 45% angles in order to capture light that escapes the sides 812 and 814 of the bulb 800 and send it directly forward and parallel to the light that is being emitted by the main face 804 of the bulb 800. Spaced as closely as possible to the two side reflectors 926 and 928 and the front electrode 902 is the UV filter plate 906 which is made of polished quartz and plated layers of Hafnium Oxide that form a high pass filter above 230-234 nm range. Ideally these layers would be applied dynamically as to completely filter the harmful wavelengths and ignore the very low levels of UV C for a much more efficient filter, possibly only 60 layers and less than 80 layers.

A heatsink 910 which could be aluminum but ideally would be ceramic to add electrical insulation to the high voltage back electrode 904. The heatsink 910 will block any unfiltered light from emitting through cracks between the mirrors 908, 926, and 928 or out of the ends of the bulb 800. Ideally the heatsink 910 would also capture many of the individual elements of the bulb assembly mentioned so far including the bulb 800, the rear reflector 908, the side reflectors 926 and 928, the rear electrode 904, the front electrode 902, and the front filter plate 906 and it would be tightly sealed using UV compatible epoxy would be used around the edges of the bulb 812 to stabilize the mechanical connections between these components and completely seal air and dust incursion. Existing art designs allow for air to be blown directly over the bulbs and dust could then deposit over time to the bulbs and the inside face of the filter. Dust can absorb large amounts of the UV C light and become very inefficient very quickly. The inventive device eliminates these faces from dust incursion by making a sealed cartridge 900 using the end caps 912 and 914. These end caps 912 and 914 of the bulb assembly 900 will be made of ceramic and the end cap 912 would encapsulate thermal sensors and a smart chip 916, as well as provide a mechanical rotation point for the bulb including detents 918 for preset individual position stops in a fixture. This means a light emitting cartridge that has no wires or flying leads to connect. The smart chip and temperature sensor 916 has an hours of operation meter, serial number, manufacturing date, temperature, out of range flags, and encryption communication capabilities to prevent counterfeit operation. There as a conductive jumper 930 in connection with the front electrode 902 that passes through the ceramic end cap 912 and then is electrically connected by to a conductive pin 920. Similarly, there is a conductive jumper 932 that is in connection to the rear electrode 904 that passes through the ceramic end cap 914 and then is electrically connected to a conductive pin 922. There are 3 plated-on conductive traces 924 around the ceramic end cap 912 that are connected to the smart chip and thermo sensor 916. These traces 924 allow communication from the bulb cartridge 900 to contacts on the fixture receiver to allow the fixture to communicate with these chips 916. Such mechanical and electrical connections are well understood by one skilled in the art and other methods of connectivity could be used. This assembly becomes an easy to replace safe UV C bulb cartridge 900 that is hermetically sealed with all high voltage portions insulated and removed from those who handle it or are exposed to it.

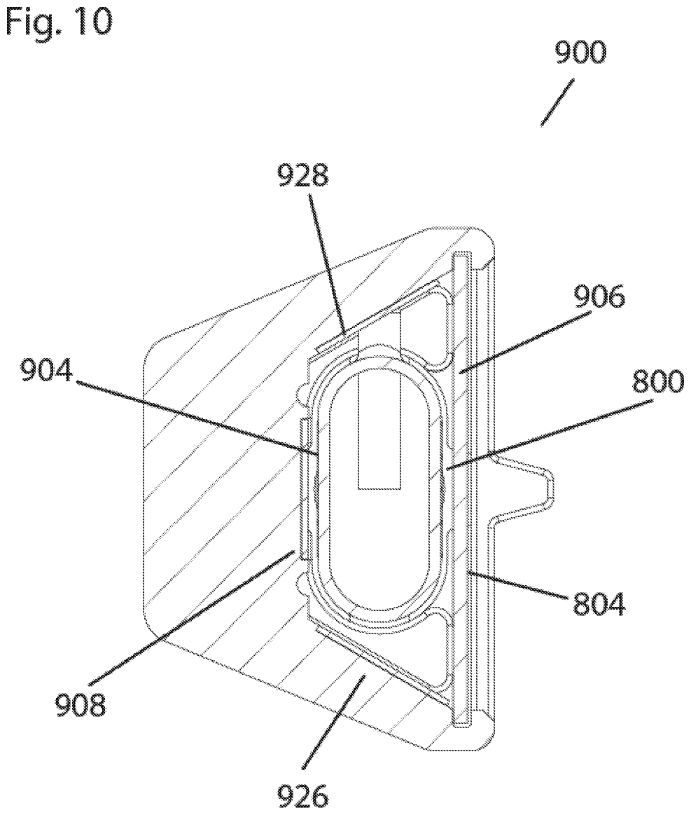

FIG. 10 depicts a sectioned side view of an assembled safe UV-C bulb cartridge 900. The bulb 800 has the negative electrode 904 plated or applied directly to the quartz. Similarly, the positive electrode 902 applied directly on the bulb's 800 front face 804. Behind the negative electrode 904 is the rear reflector 908. The right-side reflector 926 and the left side reflector 928 are angled at 45 degree angles to the front face 804. They are all captured and contained by the ceramic heatsink 910. There is a tiny air space between the bulb 800 and the filter 906 that is sealed by the ceramic pieces and ideally UV tolerant sealant.

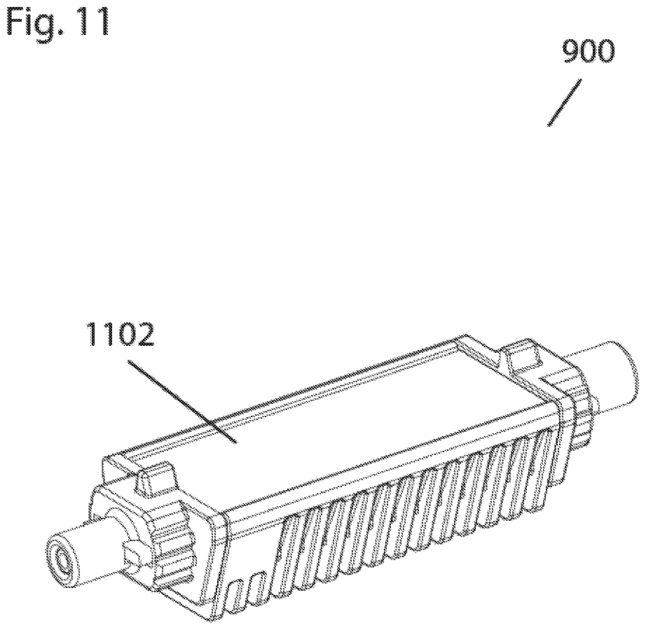

FIG. 11 depicts an assembled safe UV-C bulb cartridge 900. The assembled cartridge 900 has an optical aperture 1102 that is sealed around the edges such that air and dust and unfiltered light are eliminated from passing through.

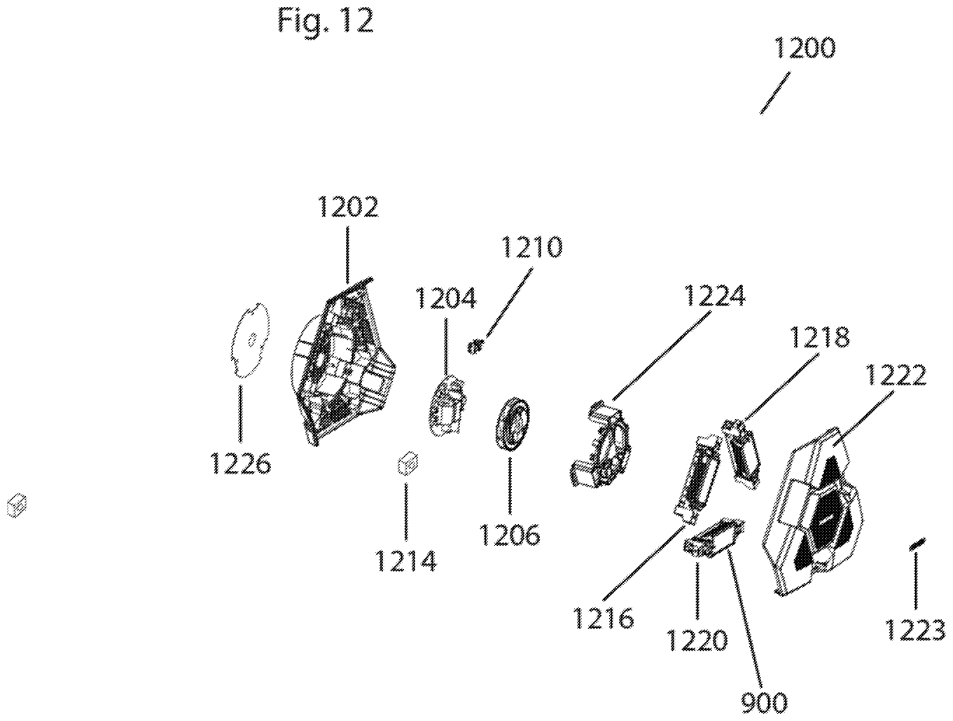

FIG. 12 shows an exploded view of a multi-cartridge variable beam angle fixture 1200. Each of the 3 bulb cartridges or heads 900 ride in ceramic saddles 1218 that have conductive spring clips 1220 that retain the metallic pins 920 and 922 of the bulb cartridges 900 as well as connect power to bulb cartridges 900. The saddles 1218 and corresponding end caps 912 at one end are a different size than the saddle 1218 and end cap 914 at the other end which allows polarizing the ends so that the bulb cartridge 900 can only be inserted one way.

The saddles 1218 also hold detent springs 1216 which mate with detent ridges and groves on the bulb end caps 912 and 914. This allows the bulb cartridges 900 to have several exact angles that they can easily be set to, the spring 1216 holding them 900 in each position but allowing finger pressure to allow it to snap to the next detent position. The front bezel 1222 of the fixture swings away from the base 1202 by means of a latch and hinged connection 1208 between the front bezel 1222 and the rear housing 1202 to expose the bulb cartridges 900 for maintenance or replacement. When the front bezel 1222 is closed completely it presses against a safety switch 1210 which is mounted in the rear housing 1202, the pressed microswitch 1210 then enables power to the fixture 1200. Proximity and distance checking is also determined by distance sensor 1214 which looks through a small hole in the bezel 1222 and checks distance to the closest object or floor. The safety switch 1210, distance sensor 1214, motion sensor 1214, light level sensor 1214, and data from the 3-bulb cartridge's smart chip and thermo sensors 916 are all connect to and coordinated by the smart power supply 1204. The power supply 1204 also has digital communication capabilities such as Wi-Fi and Ethernet to name just a couple. Air is pulled through perforations in the front bezel 1222 by a fan 1206 that is supported by a fan frame 1224 then blows this air over the top of the power supply 1204 and over the bulb cartridge's heatsinks 910 and out through holes in the base 1202. The power supply 1204 measures bulb cartridge 900 temperatures and modifies the fan 1206 speed for optimum efficiency of the bulb cartridge 900 efficiency. A mounting plate 1226 is capable of mounting first to standard electrical boxes found in existing architectural situations and then the plate snaps to the rear housing and can spin in the rear housing tracks to allow the upper bezel to aim in infinite directions. Because UV C light filters tend to allow light to only pass at narrow angles the emitted light tends to be in a narrow beam. This inventive fixture allows for multiple heads in a single fixture to allow for wider beams and asymmetrical light dispersion to best fit the widest range of environmental confines. Ideally the fixture would have an illuminated indicator 1228 to show functions and or faults from a distance, in the illustration the indicator 1228 is a backlit logo. The preferred embodiment shows 3 bulbs in a fixture but any number of bulbs could be used in the inventive device. The advantage of having more than one bulb emission angle allows for better isotropic performance. That means more sources of light coming from different angles kills pathogens better in a dynamic environment than a single narrow beam of the same total power, even if the inventive multiple light system 1200 could not swivel, even if the lights were a tight beam and not diffused, it would be superior to a single bulb angle system.

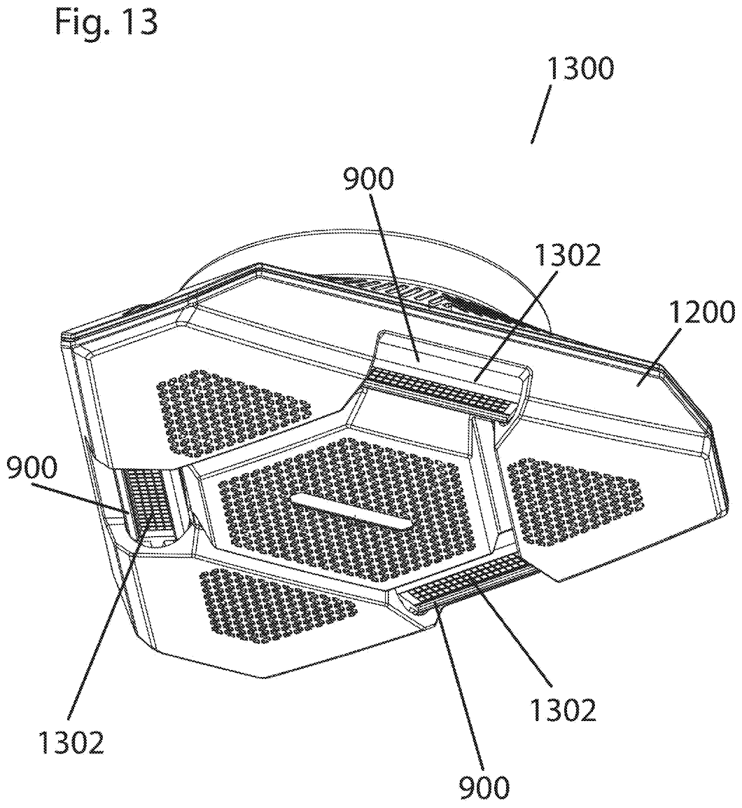

In FIG. 13 Depiction of fixture aiming down with bulbs positioned straight down 1300. Fixture 1200 has 3 bulb cartridges 900 aimed straight down 1302 for a tight beam angle under the fixture 1200. This forms the narrowest beam possible for the fixture and would be used in situations where there is a very high ceiling or where the emitted light needed to be concentrated.

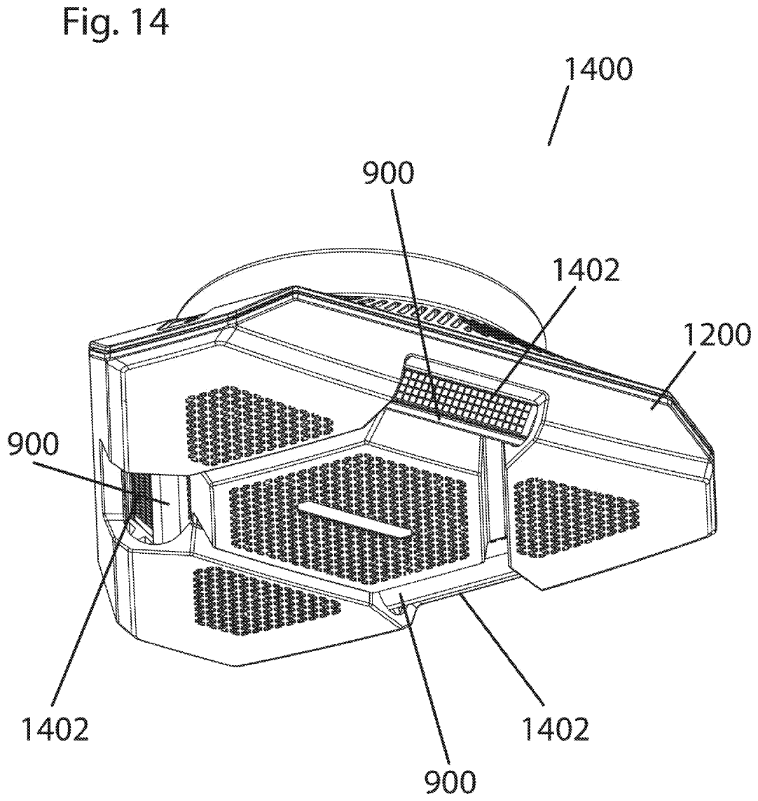

In FIG. 14 Depiction of fixture aiming down with bulbs positioned at 45 degrees 1400 where fixture 1200 has 3 bulb cartridges 900 aimed out at 45 degrees each 1402. The wide beam angle under the fixture 1200 and would be used on lower ceilings or in areas with a very wide area to be covered.

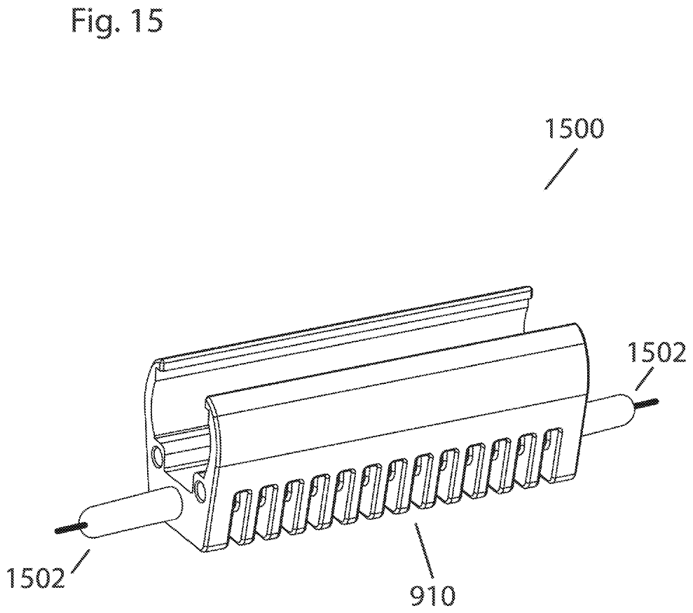

In FIG. 15 Depicts a safe bulb heat sink with a heating filament 1500. The heatsink 910 can be modified to have a heating element 1502 that is permanently installed through a hole in the heatsink or is simply embedded in the ceramic along its long axis. This heated heatsink 1500 will conduct heat to the bulb 800 and warm the gases inside making them able to ignite when requested. Electrical heating elements 1502 are over 100 years one hundred years old and well understood by one skilled in the art. This heating element 1502 is controlled by the smart power supply 1204 when needed to start the fixture 1200 during cold conditions. Once the bulbs 900 are operating they warm up and the heating element 1502 can be turned off. Extra electrical contacts would be required for this inventive addition to the excimer bulb assembly.

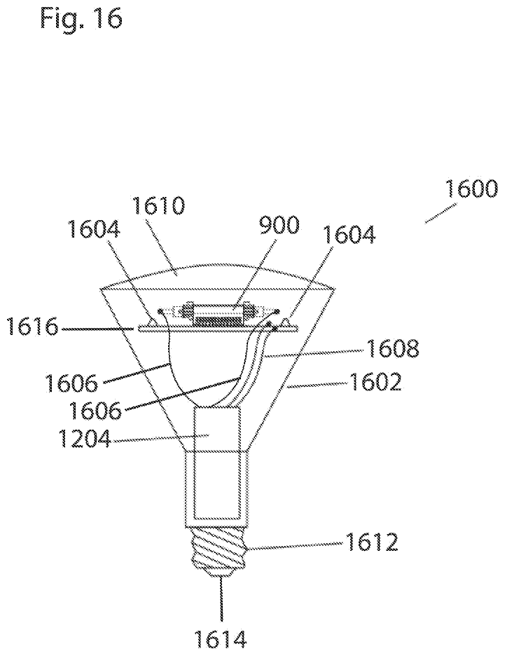

FIG. 16 depicts a side view of a screw-in UV-C safe bulb 1600. For means of illustration only this depiction uses a flood light type bulb 1602 but any type of screw in bulb or cartridge-like bulb would be applicable regarding this disclosure and this disclosure is not meant as a limitation in any way, just one example. The safe bulb with both UV-C and general illumination elements 1600 is placed inside a screw-in bulb housing 1602. The traditional electrical contacts of said screw-in bulb 1612 and 1614 or Edison screw 1612 and 1614 or mogul base 1612 and 1614 adapter go to the driver/power supply 1204. The driver/power supply 1204 then powers the UV-C portion 900 of the safe bulb with both UV-C and general illumination elements 1604 through wires 1606 on circuit board 1616 the wires go to clips mounted on the circuit board, the clips were not shown for simplicity. The driver/power supply 1204 also powers separately the white LED 1604 portion via wires 1608. The translucent face 1610 of screw-in UV-C safe bulb 1600 needs to be made of quartz glass, ideally with a diffused surface such as sandblasting or other texture and could snap on or snap off depending on the shape of beam needed. UV-C would be absorbed by any plastic or traditional glass cover. The inventive flattened cylinder bulb 900 is combined on a circuit board 1616 with a number of LEDs 1604. The LEDs could all be one color of white or they could be a mix of colors or different color temperatures with individual colored LED 1604 control to mix LEDs 1604 of different colors. The color of light emitted by just the safe UV-C portion of this inventive device will not be optically bright to the human eye, and it will also include a pinkish purple cast. The white LEDs could be pink or purple deficient so that when both the safe UV-C and white LEDs are turned on, the combined light would have a neutral color spectrum. A drop-in or screw-in can based light fixture 1600 would ideally spread its light widely using multiple bulbs and filters aiming in multiple directions and or use light modifiers all integrated as a single fixture 1600. The electrical connections could be flying leads 1612, 1614 if necessary. This bulb/fixture 1600 could be used in elevators, and other close spaces like bathrooms, hallways and such. It could also support different bezels to best fit with any given architectural style. The bulb could be adapted to fit in existing 4″ or 5″ or 6″ ceiling cans or holes and these cans or holes could be round or square, just for example and not of limitation. Similarly the bulb 1600 could be included in a troffer style fixture that is used in suspended ceilings and would ideally include LED 1604 lights for general illumination. The Far UV C bulbs 900 for a troffer would ideally be elongated rather than short. This inventive bulb 1600 could also be adapted to connect to existing ceiling-based track lighting. This would allow unique aiming and concentrating of light using multiple bulbs 1600 for specialized situations.

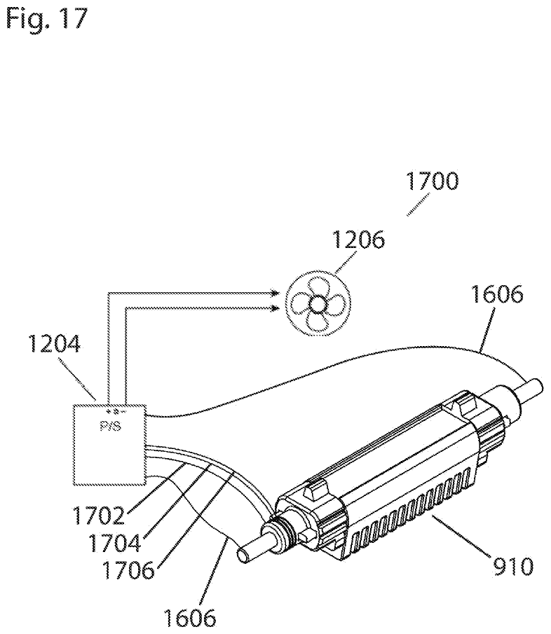

FIG. 17 depicts fixture with a temperature regulated fan-cooled flattened cylinder safe UV-C bulb fixture 1700. The smart power supply 1204 is in communication with the smart chip 916 in the bulb 900 through the low voltage data lines. Above the heatsink 910 is a small muffin fan 1206 that blows down on the heatsink 910 and bulb 900. The fan is controlled and powered by driver/power supply 1204. The thermal sensor 916 encased inside the ceramic base is interrogated by the smart driver/power supply 1204. Based on the power being sent to the bulb 900 and the reported temperature the driver/power supply 1204 will drive the fan 1206 to an appropriate speed in order to regulate the temperature of the bulb 900 in a closed loop. The heatsink 910 may or may not be necessary because lower powered bulbs would not need the heat sink 910, higher powered bulbs might need a big heatsink or a pin-fin heatsink 910, by example and not by limitation. The driver/power supply 1204 will also power resistive heaters when the bulb 900 has not been on and the thermal sensor detects that bulb ignition might not be possible due to low temperatures. The driver/power supply 1204 would then power the resistive heater before applying power to the bulb 900. Later once the bulb 900 was operating the driver/power supply 1204 might have to power the fan 1206 to cool down the bulb 900. The power supply powers the excimer bulb using pulse wave high voltage DC power in the inventive fixture. The width of the individual pulse waves can be altered to control brightness of the bulb 900 or some of the pulse waves can simply be skipped to dim the light's output. Most excimer bulbs are presently driven by sine wave power supplies and they have too much un-harvestable energy below 9,000 volts. This energy simply heats the envelope and doesn't generate light. The pulse wave may have a bit of a rounded top but otherwise looks like a square wave, it has straight sides, no unusable power. The bulb's predicted output over time can be programmed into the power supply so that as the hour meter in the bulbs age and report, the power supply could raise the power slightly to compensate for the lower efficiency to have a constant lumen output over time.

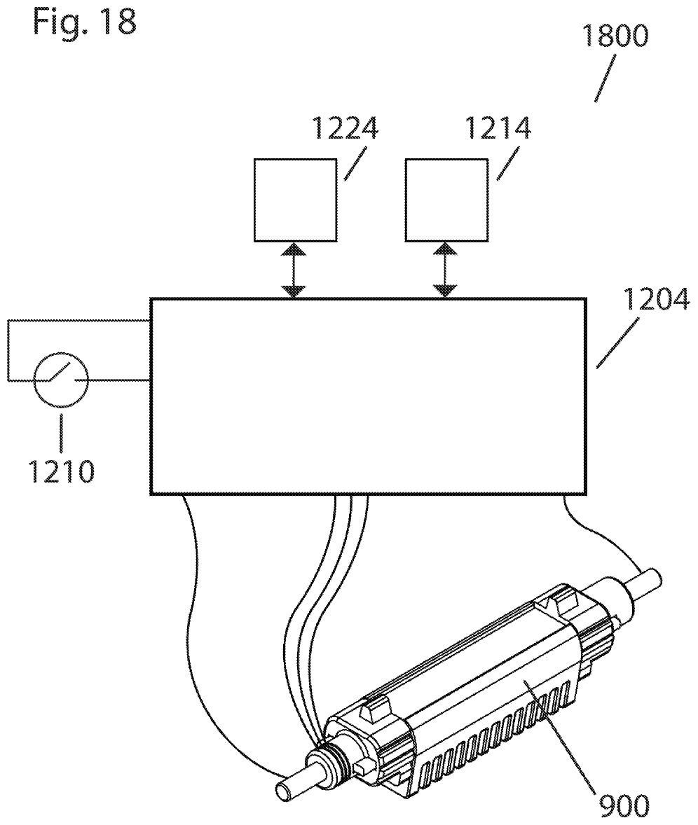

FIG. 18 is a block diagram of driver, bulb 900, distance and proximity sensor 1214, light level sensor 1224, smart chip 916 and safety cutoff switch 1210 in a fixture enclosure. The bulb 900 is connected to the driver/power supply 1204. The driver/power supply 1204 has a switch 1210 wired to the door or front bezel 1222 of the fixture enclosure so that whenever the door 1222 is open, power to the bulb 900 is turned off. This is an especially important safety issue because excimer lights can operate using several thousand volts. The driver/power supply 1204 talks to the smart chip 916 and verifies that this bulb 900 is valid and has not been tinkered with and is a valid replacement via communication with the smart chip. Counterfeit bulbs would most likely not have the pass-filter and would emit dangerous wavelengths towards the user. The distance/proximity sensor 1214 makes sure that the output power level is appropriate for the distance to the ground and could change the output power lever to the bulbs based on the distance. The proximity sensor 1214 and smart power supply 1204 look to lower power levels or turn off if it detects objects at very close distances such as a maintenance person trying to work on the fixture 1200. The proximity sensor and distance sensor 1214 could be the same sensor. The UVC light level intensity sensor 1224 could either look at the ground or at the bulb. It would be filtered to only see 200 nm-230 nm wavelengths. It could determine total light output and the smart power supply/driver could use this information to adjust the output for constant lumen output. Smart functions of the power supply 1204 could be moved to a separate circuit board and that board could then control the power supply 1204. Such functions are well known by one skilled in the art.

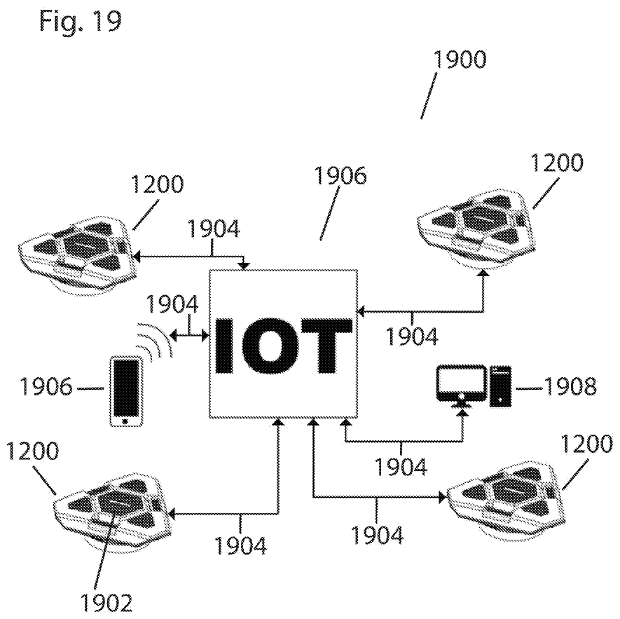

FIG. 19 Is a network diagram of IoT bulb and system 1900. The inventive device 1200 may have LEDs 1228 on the basic light fixture 1200 that will indicate that it is on and disinfecting. This could be a constant LED 1228 or intermittent LED 1228. Similar to a smoke detector, the light 1200 will be “good” or “OK” in one color IE Blue or Green when functioning properly and will turn to “attention” or “error” in another color IE Amber or Red. The indicator 1228 could be in the form of a backlit logo.

People entering a room can quickly look and see that the light 1200 is functioning or needs maintenance by looking at the LED 1228 or LCD 1228. The led 1228 may also indicate output level. When there is low activity and low bacterial load events it could have one LED on, when there is high activity with increased bacterial load events, the light increases output and the LEDs 1228 will change to signal this event. The fixture could receive data from crowd density sensors and use this information to set the output power levels.

Crowd density sensors 1906 such as CrowdScan 1906 an rf monitor from Antwerp or Density 1906 which is a Lidar based device from San Francisco have the ability to determine how many people are in a given space at a given time without violating their privacy, i.e. using cameras or cell phone snooping techniques. There are several more services similar to these which are simply examples of crowd density sensors 1906 that communicate as IoT 1906 and internet resources such as the inventive device 1900.

The light 1200 may integrate several different kinds of communication 1904 to include BlueTooth 1904, WiFi 1904, Cellular 1904, Sidewalk 1904 from Google, and hard-wired technologies 1904 to mention a few. This communication 1904 will allow for the monitoring of the light function and allow remote control of the light by remote means.

The light 1200 may have local mechanical control systems such as simple on/off and dimmable light switches. The light(s) 1200 may also have a control panel with switches and LEDs to control many lights. The LEDs in the panels can show status or light (on, off, status, etc.). The inventive device lights 1200 can be integrated with other traditional visible/functional lighting. These physical controls would allow controls over those lights also. Physical controls can vary depending on the light fixture application. For applications the fixture 1200 is installed permanently in a space the controls can be integrated into the facility infrastructure. For stand-alone portable applications the controls may be fully integrated into a light 1200 to include integrated power source with power level indicators and a graphic user interface display and control panel.

The light fixture 1200 can communicate 1904 to facility/installation managers and operators. The information can be accessed by a smartphone application 1906, web interface on a laptop 1908 or desktop 1908. The fixture 1200 will push information to the site and the operator can pull information from the light 1200. The wireless interface can be customized for different users' needs. The light 1200 can communicate what output level it is at, what the energy consumption level is, internal temperature, lifecycle/hours the bulb 900 has been in use and how long till it will need to be replaced, work in combination with motion detection to determine if there is a high bacterial load in the space it is set up in. The operator can also control the level of the light 1200 output and schedule the operation profile customizable to best sterilize the area and optimize energy consumption.

The light fixture 1200 can communicate the status to the public or space occupants. The information can be accessed by a smartphone 1906 application, web interface on a laptop 1908 or desktop 1908. This will reassure occupants that the space is being sterilized. The information can also be displayed on an information display in the space.

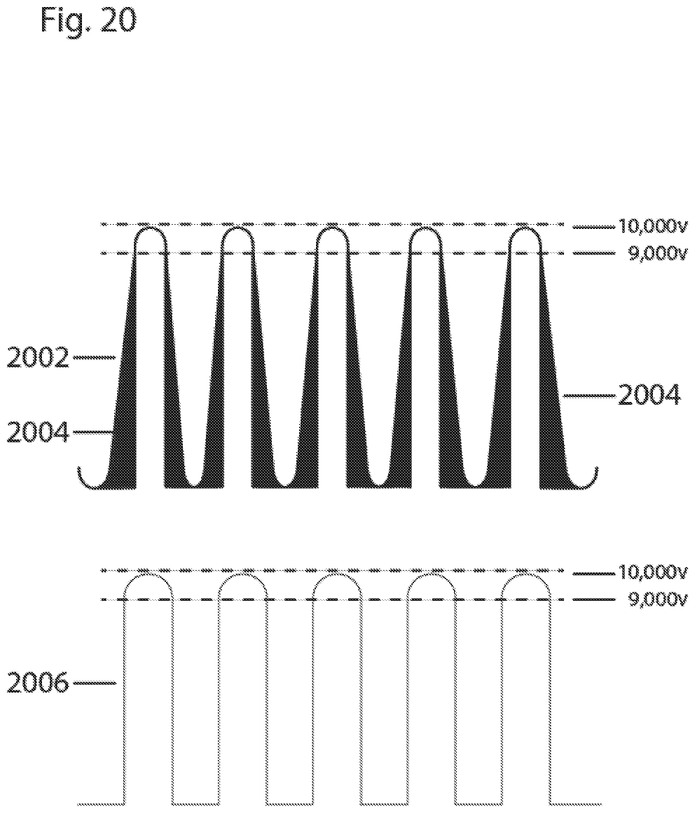

FIG. 20 is a diagram showing a couple of the power pulse trains used for driving excimer bulbs and showing the differences between 10,000 volt sine wave 2002 power compared to 10,000 volt pulse wave 2006 power. Only the top voltages between 9,000 and 10,000 volts which are shown in the light areas make usable light. All voltages below 9,000 volts simply make heat and that heat minimizes the power level that a bulb can be driven to. The dark area 2004 between the outline of the sine waves and the center white areas are all wasted power and turns to heat that is generated inside the bulb that does not come out as light. Heat causes the breakdown of chlorine gas and that dictates a shorter lifetime of the bulb. There is no wasted voltage in the pulse wave power supply example. The inventive device 1200 would ideally use pulse wave power 2006 in its power supply 1204. This should provide a 50-100% increase in bulb efficiency over the existing sine wave based UV C fixtures. It is also possible to lower the voltage of the pulse wave 2006 below 10,000 volts to dim the bulb's 800 output. The voltage range based on the supplied graph would be 100% at 10,000V and 0% at 9,000V, which is a very narrow voltage range that has to be modified and controlled.

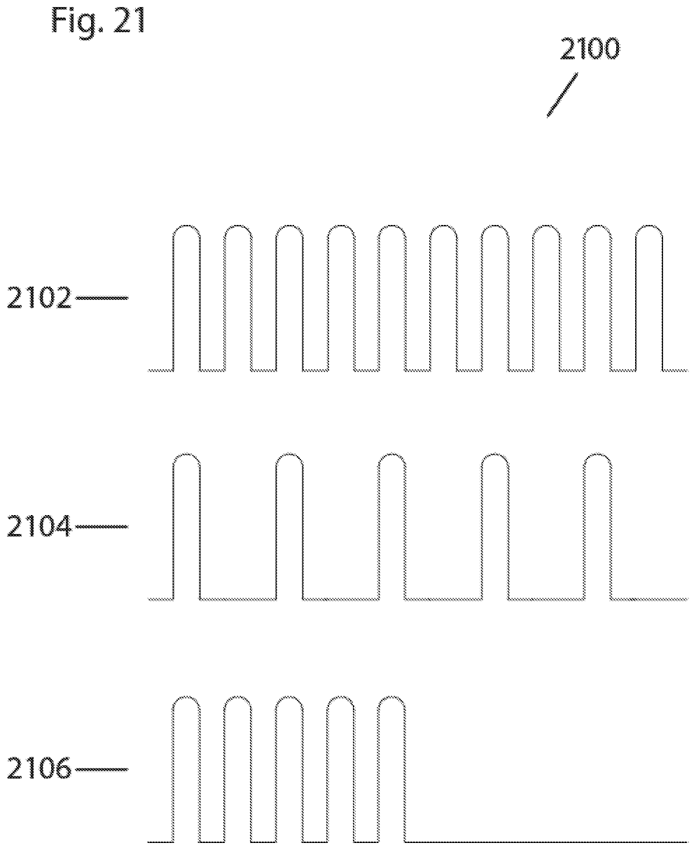

FIG. 21 is a diagram showing the power pulse train 2100 for driving excimer bulbs where the pulse wave 2102 is at 100% and a pulse wave 2104 at 50% dimming using a symmetrical pattern by reducing every other wave, and a pulse wave 2106 at 50% dimming using an asymmetrical pattern. The power supply simply has to remove individual pulses to reduce brightness and because the pulses are so fast, between 10 k Hertz and 250 k Hertz small dropouts are not obvious and the amount of visible light generated is negligible even at 100%. The smart power supply has a microprocessor that subtracts some of these pulses to dim the fixture's output to any level down to 1% or lower. This technique is well understood to those skilled in the art. These graphs are for examples only, the actual voltages will vary with bulb design and internal gas compositions.

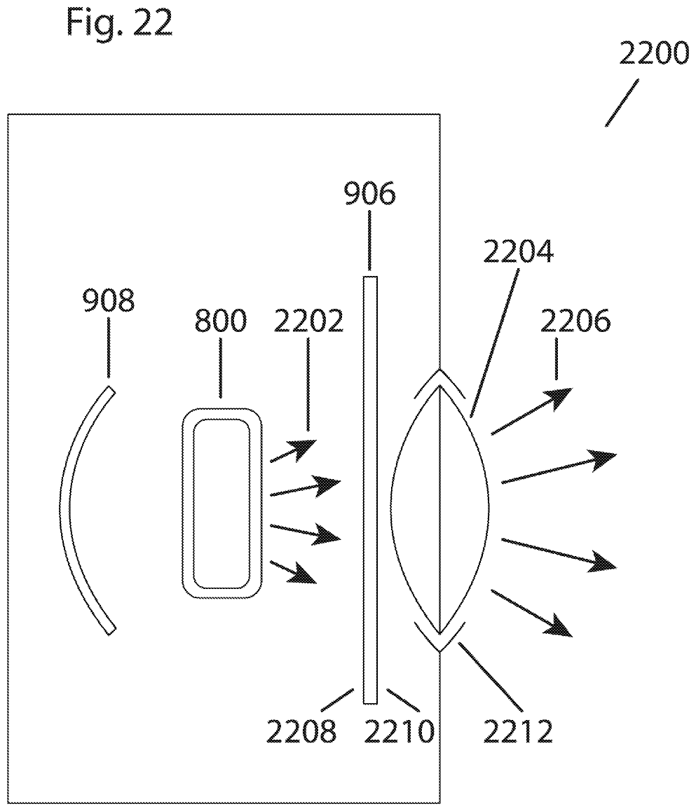

FIG. 22 Shows a side view depicting an excimer bulb 800 with variable focus as a system 2200. The bulb 800 has positioned in the light path 2202 a quartz lens 2204 with light shaping capability. The lens 2204 can be round and symmetrical or linear to best match the initial optical path 2202. The lens 2204 could also be continuous or a Fresnel which is stepped. The lens 2204 can be moved closer or further to the excimer bulb 800 to change the focus and spread or narrow the beam angle of the finally emitted light 2206. The light modifying element 2204 which is in the light path 2202 but positioned past the filter 2208 could be a layer of diffusion or a reflector that is designed to spread or shape the light output 2206. This light modifier 2204 could be directly part of the filter 2208 or a lens or aperture. The variable focus system including a reflector 908 or 2204 which would ideally be curved and it could be moved closer or further from the bulb 800 to change the angle of light emitted from the system 2200 and the reflector 2204 could have perforations and multiple curves for asymmetrical light shaping. The reflective light modifier 2204 could be tilted, flat, parabolic, compounded or a combination of these. It 2204 could be perforated, dimpled, or partially sandblasted. Because the filter 2208 tends to make a narrow beam of light, the lens 2204 could be replaced or supplemented with a diffusion material 2210 on the outside of the quartz filter glass. The light modifier 2204 could be placed on the outside of the filter quartz 2210, and could be milky, sand blasted, or embossed patterns, pickling, chemical etching in the glass 906 or 2204, glass balls, nano particles, or any translucent material that would spread the light 2206 without too much subtractive loss. There are reflective materials that are specifically designed for UV such as aluminum but the most efficient is Teflon which can exceed 95% reflectivity. The problem with Teflon is that it scatters the light that is reflected, it is not directable as polished aluminum.

Ideally the light modifier 2204 would be mounted at an attachment point on the outside of the bulb assembly 2212 or fixture 2212 in such a way that it could be easily removed and replaced with a different light modifier 2204 with different optical characteristics 2206. The attachment means could include using magnets, clips, Velcro, springs, indentions, slides, or screws simply as examples, not limitations. If the bulb 800 was not an excimer-based bulb but a UV LED or a UV C LED it could similarly use lenses or other light modifiers to shape the light 2206. UV LED PCBs could be manufactured with different light modifiers 2204 integral to the PCB so that different PCBs would be used for different radiation pattern 2206 situations. The UV PCBs would be exchanged in a given fixture. One skilled in the art could easily find an attachment method to best match the unique physical characteristics of the final embodiment of all these technologies. One of the most important features of diffusion is that as a light modifier 2204 it mixes the light. The filters 2208 used in the present invention cause a central pattern 2206 of safe light surrounded by an annular ring of low intensity light that is close to unsafe wavelengths. The diffusion 2204 spreads the safe light from the center but also spreads the annular ring, lowering the intensity of the undesirable wavelengths even further.

The light modifiers 2204 listed above could be used in a fixture 1200 that would perform upper air disinfection. The requirements for upper air disinfection include high levels of broadly aimed 2206 UV radiation above the height of a human head in the environment to be disinfected. Also very low levels of UV below the height of a human head in the environment to be disinfected, regardless of the reflectivity of the ceiling or other architectural elements. The light modifiers 2204 for excimer bulbs or UV LEDs would be shaped or positioned to best achieve upper air disinfection even if the pattern 2206 is asymmetrical. The shape and dimensions of the room as well as the ceiling reflectivity would largely dictate the light modifiers' 2204 shape and target. A hallway upper air disinfection system would obviously direct energy laterally 2206 in opposite directions for a long narrow pattern 2206 going both ways. A system deployed in room that was square would have a much more omnidirectional pattern 2206. A room with a highly reflective ceiling would need a pattern 2206 of UV that was blocked from reaching the ceiling and mostly directed outwardly, it would be shallow and pancake shaped. These are a few examples of targeted shapes, but the present invention is not to be limited by them. The LEDs could also be 405 nm, which is actually 5 nm outside the defined UV range, that would be used for horticultural disinfection.

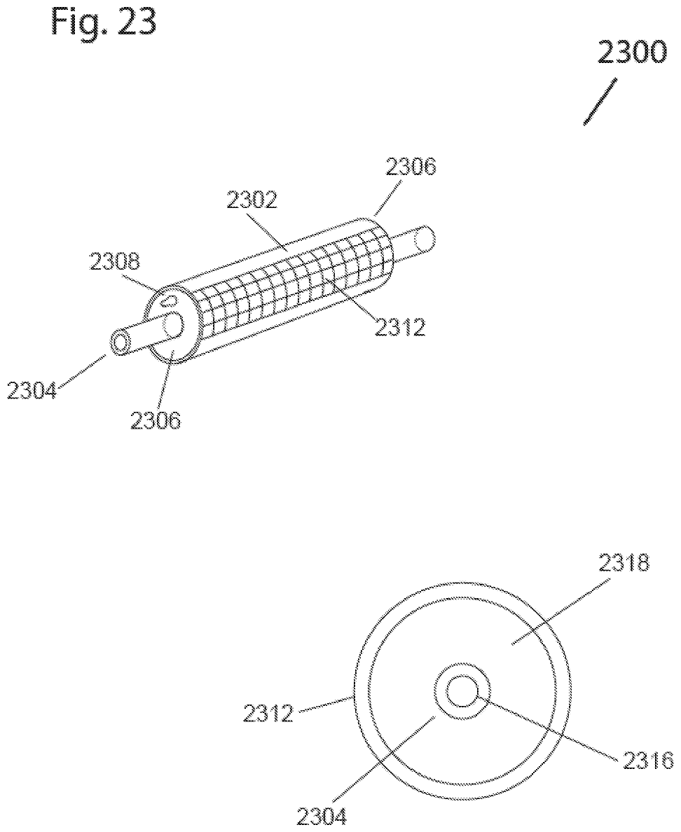

FIG. 23 Shows a liquid cooled high power excimer bulb 2300. It consists of an outer envelope 2302, and an inner envelope 2304, where the outer envelope 2302 is shorter and the inner 2304 is longer and they are connected by 2 end caps 2306 which are welded together by heat. The area between the inner and outer has a fill point 2308 on one of the end caps 2306. The fill point 2308 similar to those on existing excimer bulbs and this form chamber 2318 between the two envelopes is evacuated an filled with the appropriate excimer gasses previously mentioned, through the fill point 2308. The outer wall 2310 of the outer envelope 2304 has a conductive grid 2312 adhered to it and it becomes the negative electrode which goes all of the way around the circumference of the tube 2304. Similarly the inner wall 2314 of the inner tube 2304 has a conductive grid 2316 which goes around its inner circumference and becomes the positive electrode. Light exits equally in all directions from this type of bulb.

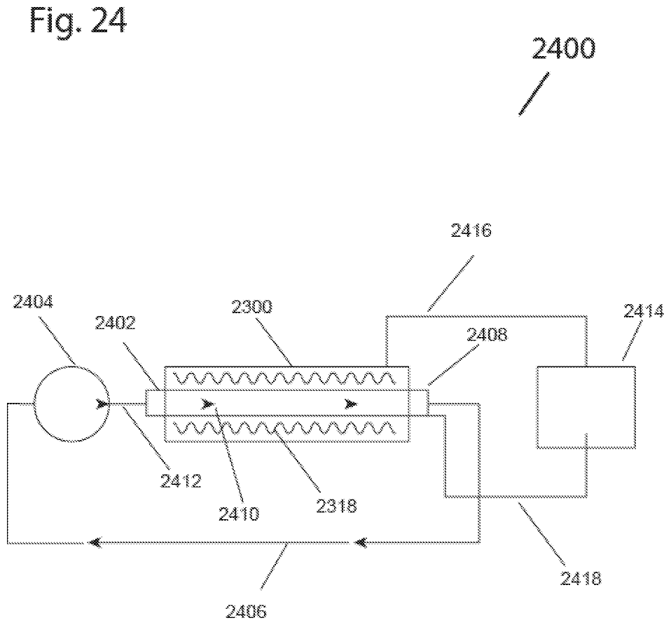

FIG. 24 Shows a block diagram for a liquid cooled bulb 2300 with the coolant system which results in a high powered excimer fixture 2400. The bulb 2300 has a tube 2412 connected to the input end 2402 that connects to a pump's 2404 output. The pump's input is connected to a return loop 2406 of tubing which goes out and around to the output end 2408 of the bulb 2300. Inside of the tubing 2406 and the pump 2404 and the inner wall 2316 is a single phase dielectric liquid which is circulated using the pump 2404. This dielectric liquid 2410 is typically used in large data centers where the entire server is submerged in a vessel to cool the server components. In general, there are two main liquid categories, Hydrocarbons (i.e. mineral, synthetic or bio oils) and fluorocarbons (i.e. fully engineered liquids). The liquid 2410 conducts heat very well but is not electrically conductive which is a very important characteristic for the inventive device 2400, the liquid 2410 will be in contact with the 10,000 volt conductive mesh 2316 of the bulb 2300. Heat is generated in the interior 2318 of the bulb 2300 and this heat is carried away by the pump 2404 and it dissipates to the environment during its trip through the return loop 2406 before reentering the pump 2404, where the recirculation continues. The return loop 2406 might include a separate radiator 2406 similar to what is used in the computer cooling industry, this is well known to those skilled in the art. The power supply 2414 is connected to the bulb 2300 using the negative wire 2416 which connects to the exterior mesh 2312. The power supply 2414 also connects to the bulb 2300 using the positive wire 2418 which connects to the inner mesh 2316 and is in contact with the coolant 2410. The cooling 2410 allows for much more power to be introduced to the bulb 2300 allowing for an unmatched and powerful excimer fixture 2400.

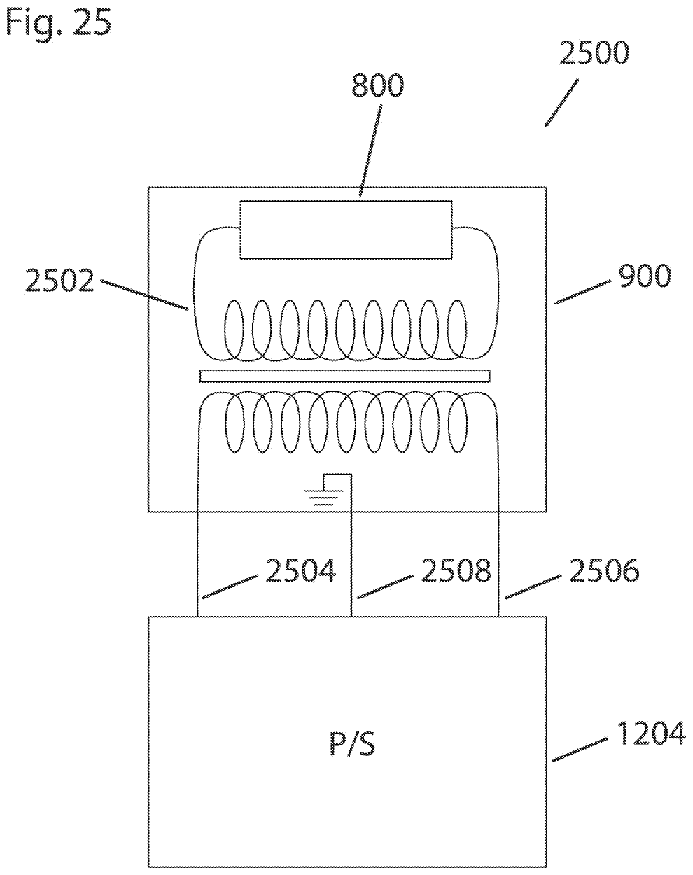

FIG. 25 Shows a schematic of a low voltage UVC bulb system 2500. The bulb 800 is driven by a high voltage transformer 2502 which is inside the bulb assembly 900. This allows the bulb cartridge 900 to be closer to surrounding structures because there are no high voltage leads to be routed and separated. This also allows the user to replace bulbs 900 without the risk of shock because the high frequency AC power delivered to the bulb assembly could be 48 VAC. The high voltage transformer 2502 has been separated from the power supply 1204, similar to what is used in flash xenon systems. This could allow for only 2 wires 2504, 2506 to be connected to the bulb assembly, a third connection earth ground 2508 could be optional.

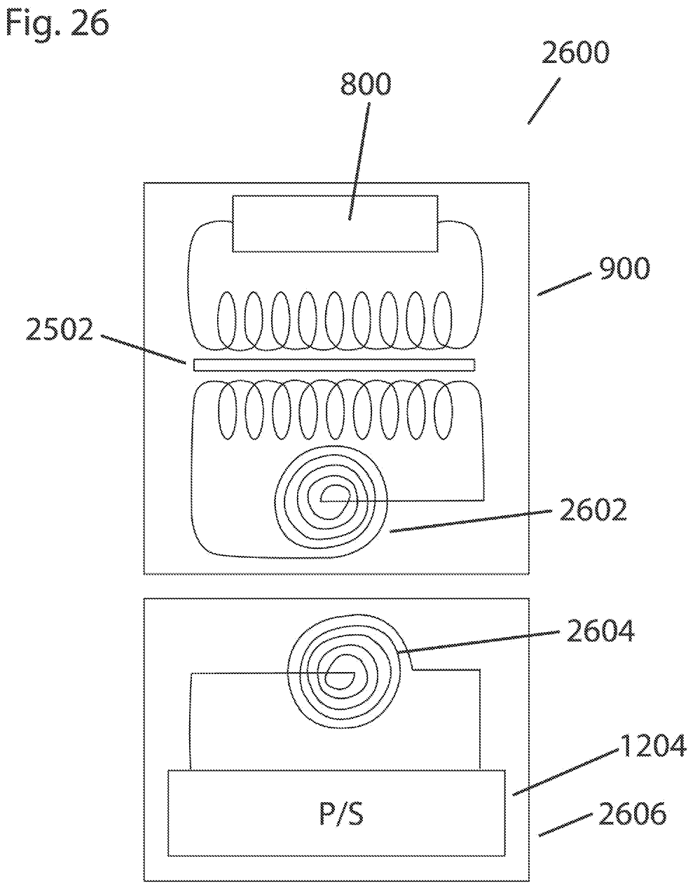

FIG. 26 Shows a schematic of a low voltage inductively powered UV C bulb system 2600. The bulb 800 is driven by a high voltage transformer 2502 which is inside the bulb assembly that is powered by a pair of inductor coils 2602, 2604. The bulb side inductor 2602 is physically located in the bulb assembly 900. The power supply inductor 2604 is located inside the power supply enclosure 2606. This allows the bulb 900 to be closer to surrounding structures because there are no high voltage leads to be routed and separated. It also removes any direct electrical connections to the bulb assembly 900. The inductor connection 2602, 2604 is similar to existing cordless cellphone charging devices, except the AC voltages used would be at a very high frequency. And because the bulb assembly 900 can be precisely cradled the efficiency of the inductive bridge 2602, 2604 would be much higher than the cell phone example. This would be the safest excimer bulb imaginable, with no mechanical electrical connections.

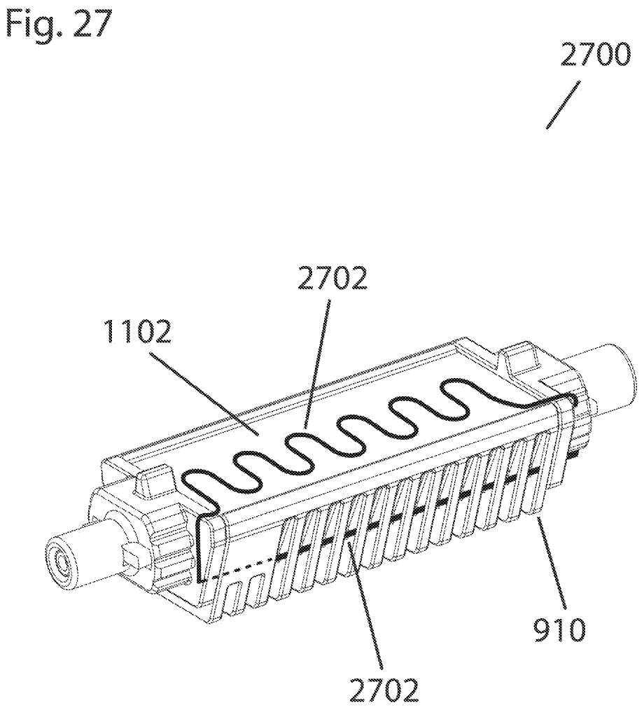

FIG. 27 Shows a isometric view of a protected bulb cartridge 2700 using a traditional bulb assembly 900 with a continuous safety wire 2702. The safety wire runs back and forth across the glass aperture 1102 on the outside and continues through or around the ceramic body 910 of the bulb assembly 900. The wire on the glass could be vacuum deposited aluminum 2702, and the wire on the ceramic housing 910 would ideally be pad printed conductive ink 2702 for ease of manufacturing and cost savings. Compare this drawing to the plain FIG. 11 to best understand the path of the wire. If the wire goes through the ceramic body 910 then it would be placed during the semi-liquid stage of the ceramic fabrication, before firing in a kiln.

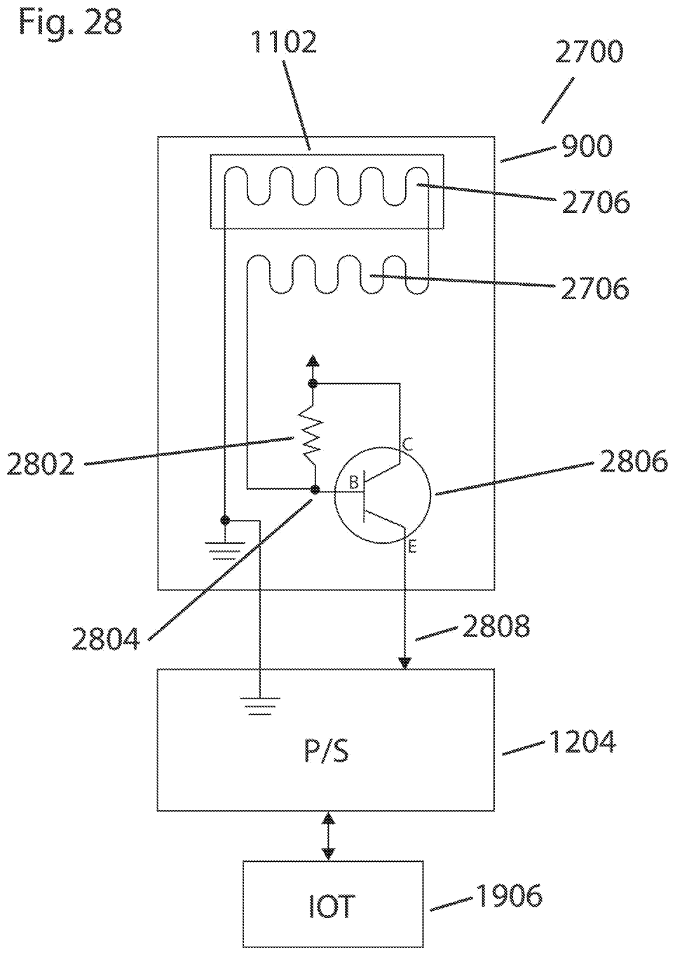

FIG. 28 Depicts a schematic of a protected bulb cartridge 2700 system using a traditional bulb 900 with a continuous safety wire 2702. The wire 2702 which could be a trace 2702, much like a conductive path 2702 on a circuit board, begins at one end where it is tied to earth ground and then routed through the ceramic body 910 of the bulb cartridge 900. The wire 2702 continues to run across the glass aperture 1102 of the bulb assembly 900. The wire 2602 then is connected to a small pull up resistor 2702 and the gate 2804 of a PNP transistor 2806. The transistor 2806 is always in the “on” condition if the wire 2702 remains intact, but if the trace 2702 is broken the resistor 2802 pulls the transistor's base or gate 2804 high, this causes the transistor 2806 to turn off, disabling the bulb cartridge 900. The transistor could electrically interrupt the power to the bulb 800 or simply send a signal 2808 to the power supply 1204, much like the safety switch 1210 does in the fixture 1200. The implementation that would use such techniques are well known by one skilled in the art and many other electrical schemes could be used other than the bi-polar transistor that was shown. This would allow fixtures 1200 to be placed to be placed in close and unsupervised proximity to people that could touch and break a bulb 900 but the act of doing so would turn off the fixture 1200. This feature could also be connected to the IOT functions 1906, alerting monitors that breakage had occurred.

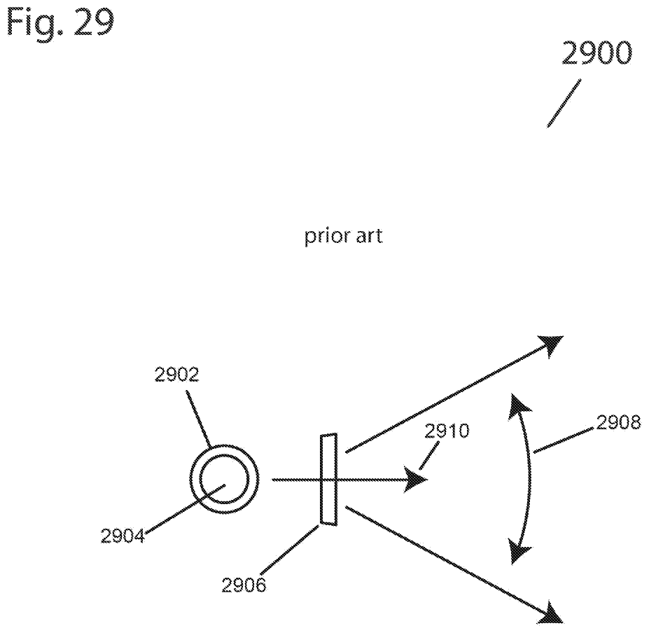

In FIG. 29, a representative depiction of an (existing art) filtered Far UV C bulb which is similar to bulb 800 and hafnium filter combination 2900. The filter 2906 is similar to the filter 906 in construction and function. The bulb's envelope 2902 contains the krypton/chlorine excimer gas 2904 that when excited generates UV C light in all directions. The bulb 2902 shown is round and cylindrical but there are many different common shapes, some are tubes that are flattened with two parallel sides (front and back) and two rounded sides, and some are tubes with a rectangular cross section, to mention a few. From the bulb 2902 the light 2910 that is generated in a line near to perpendicular to the filter 2906 passes through the filter 2906 and forms a narrow beam angle (50% of peak) with the field angle 2908 (10% of peak) being less than 70 degrees. Generated useful light that is greater than 35 degree from perpendicular to the filter 2906 is wasted because the filter 2906 absorbs all of it. This method of generating filtered Far UV C light is very inefficient.

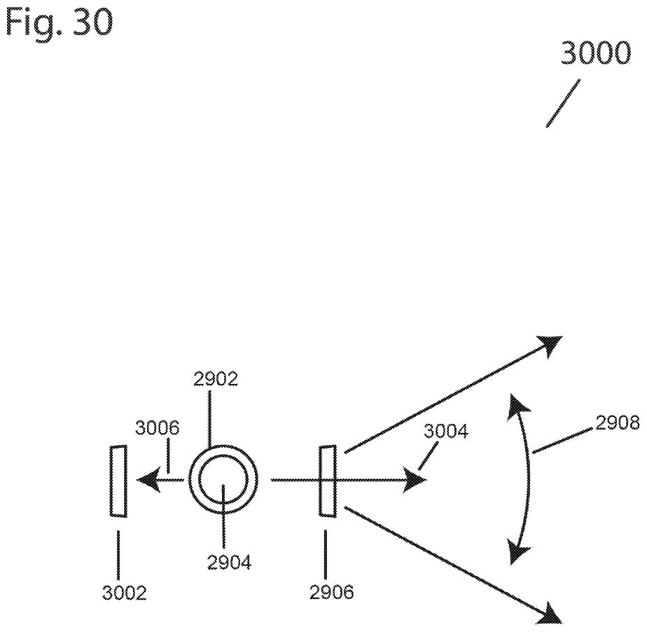

In FIG. 30 shows a depiction of a (existing art) filtered Far UV C bulb and filter with a back side mirror 3000. This filtered Far UV C is method of generating safe Far UV C light is more efficient than was shown in FIG. 29 by almost 70%. The mirror 3002 adds a useful path for the light 3006 generated by the bulb 2902 that comes out of the back side, it bounces back through the bulb 2902 and out the front in addition to the original perpendicular light 3004. There are losses in the mirror 3002 of about 10% and there are losses through each layer of the bulb's quartz envelope 2902, about 10% for each wall, for total losses of about 30%. The mirror 3002 is specially processed aluminum to be so efficient, most mirrors or mirrored surfaces would absorb most of the UV C.

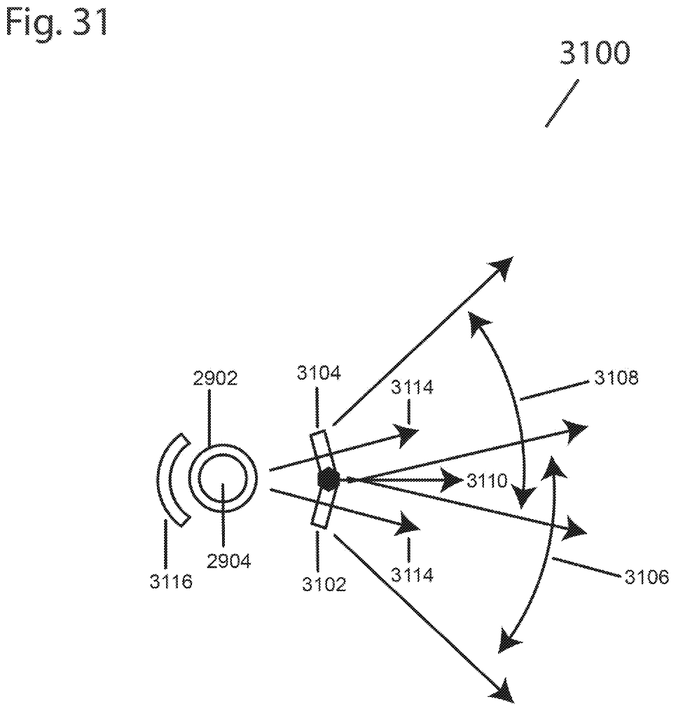

FIG. 31 shows a side section view of the inventive device, a Far UV C bulb with multifaceted filters 3100 using 2 filters. The bulb's envelope 2902 is the same as in the previously shown examples of FIG. 29 and FIG. 30. The big difference is that there are two filters 3102, 3104, each is angled to be perpendicular to the angle of the bulb's emitted light 3112, 3114. The light emitted through each filter 3102, 3104 separately has a beam, and field 3106, 3108 angles similar to what was seen in the previous single filter examples FIG. 29, FIG. 30. The filters are connected ideally together using an opaque epoxy 3110 to mechanically connect the filters and block light leaks between the filters. This inventive device would ideally have a curved mirror 3116 on the back side of the bulb 2902 to add 70% to the previous examples. The mirror 3116 is shown as a separate part but could be plated onto the bulb's envelope 2902 for greater efficiency and lower cost. Each of the 2 filters 3102, 3104 separately would have the same lumen output as the previous single filter examples FIG. 29, FIG. 30 so this geometry would have twice the total Far UV C output as the single filter versions. Twice the beam and field angles, twice the total delivered Far UV C output using the same power and bulb as before.

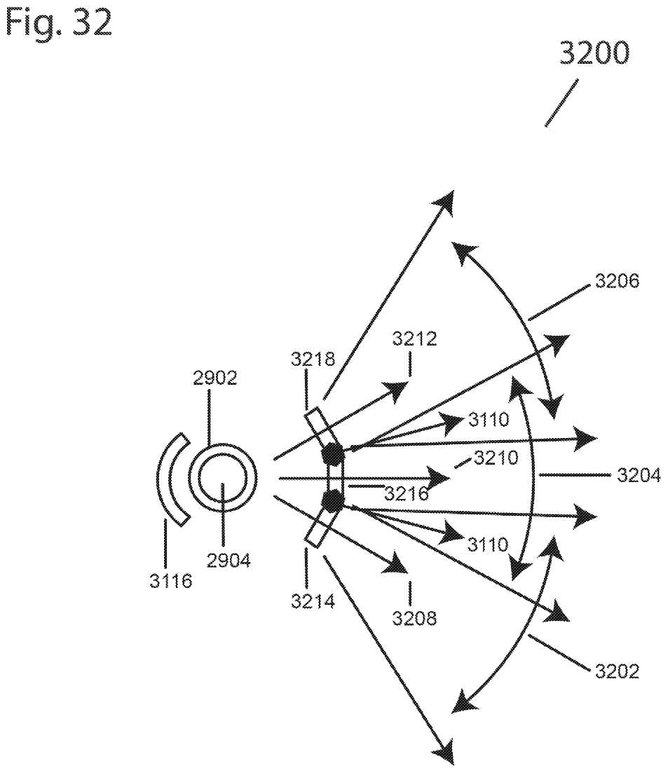

FIG. 32 shows a side section view of the inventive device, a Far UV C bulb with multifaceted filters 3200 using 3 filters. The bulb's envelope 2902 is the same as in the previously shown examples of FIG. 29 and FIG. 30. The big difference is that there are three filters 3214, 3216, 3218, each is angled to be perpendicular to the angle of the bulb's emitted light 3208, 3210, 3212. The light emitted through each filter 3214, 3216, 3218 separately has a beam, and field 3202, 3204, 3206 angles similar to what was seen in the previous single filter examples FIG. 29, FIG. 30. The filters are connected ideally together using an opaque epoxy 3110 to mechanically connect the filters and block light leaks between the filters. This inventive device would ideally have a curved mirror 3116 on the back side of the bulb 2902 to add 70% to the previous examples. The mirror 3116 is shown as a separate part but could be plated onto the bulb's envelope 2902 for greater efficiency and lower cost. Each of the 3 filters 3214, 3216, 3218 separately would have the same Far UV C output as the previous single filter examples FIG. 29, FIG. 30 so this geometry would have three times the total Far UV C output as the single filter versions. Three times the beam and field angles, three times the total delivered lumens using the same power and bulb as before.

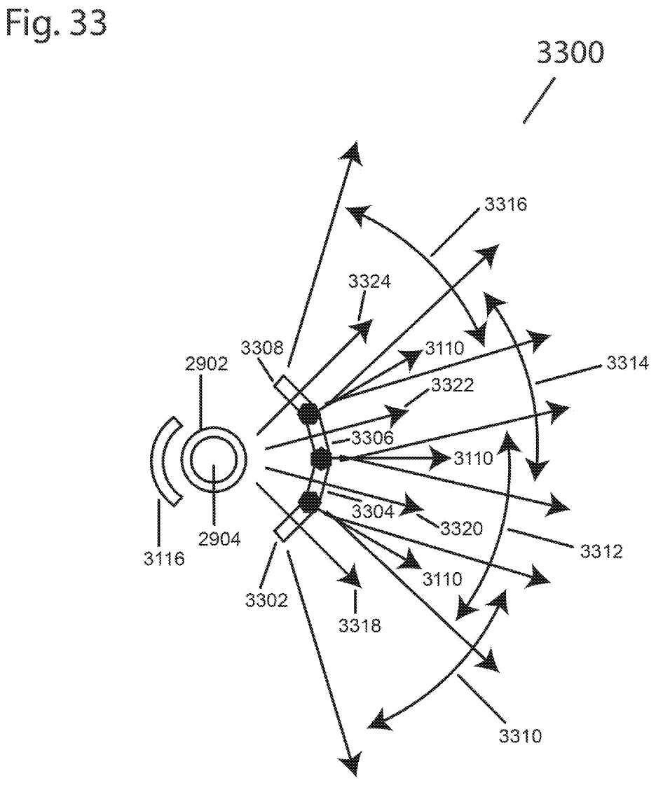

FIG. 33 shows a side section view of the inventive device, a Far UV C bulb with multifaceted filters 3300 using 4 filters. The bulb's envelope 2902 is the same as in the previously shown examples of FIG. 29 and FIG. 30. The big difference is that there are 4 filters 3302, 3304, 3306, 3308, each is angled to be perpendicular to the angle of the bulb's emitted light 3318, 3320, 3322, 3324. The light emitted through each filter 3302, 3304, 3306, 3308 separately has a beam, and field 3310, 3312, 3314, 3316 angles similar to what was seen in the previous single filter examples FIG. 29, FIG. 30. The filters are connected ideally together using an opaque epoxy 3110 to mechanically connect the filters and block light leaks between the filters. This inventive device would ideally have a curved mirror 3116 on the back side of the bulb 2902 to add 70% to the previous examples. The mirror 3116 is shown as a separate part but could be plated onto the bulb's envelope 2902 for greater efficiency and lower cost. Each of the 3 filters 3302, 3304, 3306, 3308 separately would have the same Far UV C output as the previous single filter examples FIG. 29, FIG. 30 so this geometry would have four times the total Far UV C output as the single filter versions. Four times the beam and field angles, four times the total delivered Far UV C output using the same power and bulb as before. More importantly the beams 3310, 3312, 3314, 3316 begin to overlap each other and consequently blend together. This fundamentally changes the nature of the light from a narrow beam single filter fixture into a wide beam and relatively even beam of light that is very efficient.

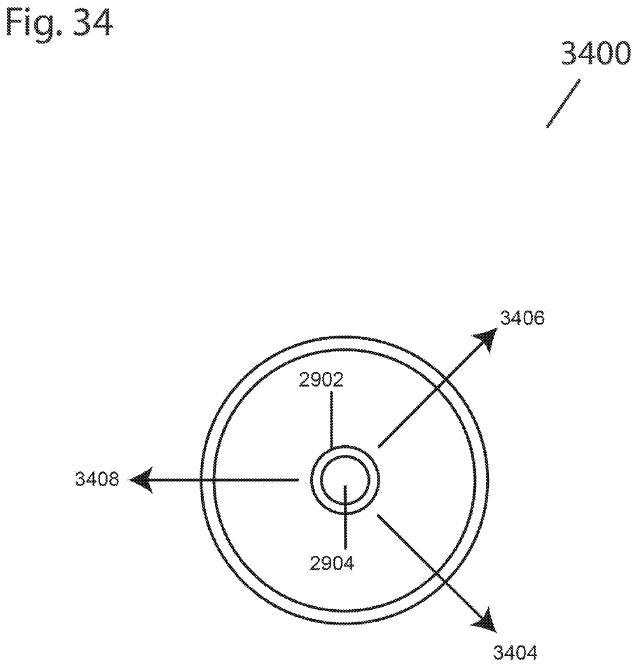

FIG. 34 shows a side section view of the inventive device, a Far UV C bulb with a multifaceted filter 3400 using 1 cylindrical filter. The bulb's envelope 2902 is the same as in the previously shown examples of FIG. 29 and FIG. 30. The big difference is that the cylindrical filter 3402 acts as an infinite number of flat filters with no epoxy joints if the bulb 2902 is placed in the center of the cylindrical filter 3402. Light 3404, 3406, 3408 being emitted from the core 2904 of the bulb 2902, no matter the angle will always pass perpendicularly through the cylindrical filter 3402. If the bulb 2902v was not placed in the center of the cylindrical filter 3402 there would only be 2 small areas where the light passes perpendicular to the bulb's light path's 3404, 3406, 3408. When the bulb 2902 is properly situated there is virtually no generated light that is blocked for not being exactly perpendicular to the filter 3402, so there are no losses except for the ends of the cylinder that must be optically sealed off. This inventive device could provide as much as ten times more Far UV C than a single flat filter fixture using the same power. Ideally the filter 3402 could be directly plated onto the bulb's outer surface 2902 for maximum cost savings.

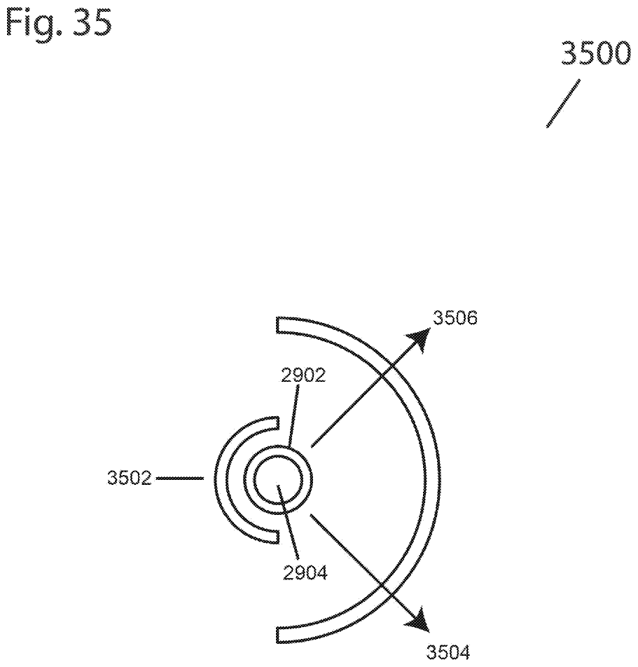

FIG. 35 shows a side section view of the inventive device, a Far UV C bulb with a multifaceted filter 3500 using a hemi-cylindrical filter. The bulb's envelope 2902 is the same as in the previously shown examples of FIG. 29 and FIG. 30. The big difference is that the hemi-cylindrical filter 3502 acts as an infinite number of flat filters with no epoxy joints. Light 3504, 3506 being emitted from the core 2904 of the bulb 2902, no matter the angle will always pass perpendicularly through the cylindrical filter 3502. The inventive bulb and filter combination 3500 also has the benefit of a mirror 3502 to further boost the light output approximately 70%. This mirror 3502 is curved in a semi-cylindrical shape on the backside of the bulb 2902. Ideally the mirror 3502 would be plated on the backside of the bulb 2902. There is virtually no generated light that is blocked for not being exactly perpendicular to the filter 3502, so there are no losses except for the ends of the semi-cylinder filter that must be optically sealed off.

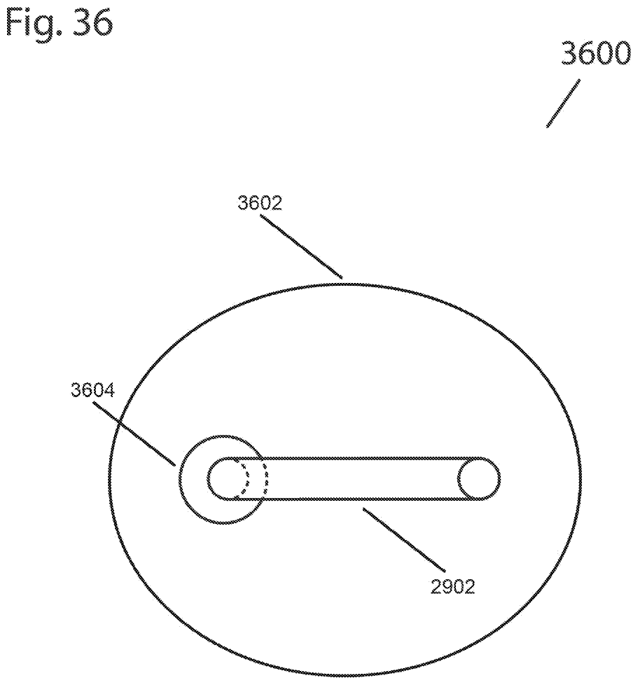

FIG. 36 shows a view of the inventive device, a Far UV C bulb with a multifaceted filter 3600 using a spherical filter. The bulb's envelope 2902 is the same as in the previously shown examples of FIG. 29 and FIG. 30. The big difference is that the spherical filter 3602 acts as an infinite number of flat filters in all 3-dimensional directions for the most efficient far UV C bulb filter combination. Light generated would be at a near perpendicular angle to the filter's face. The only losses are the entry point 3604 for the bulb 2902.

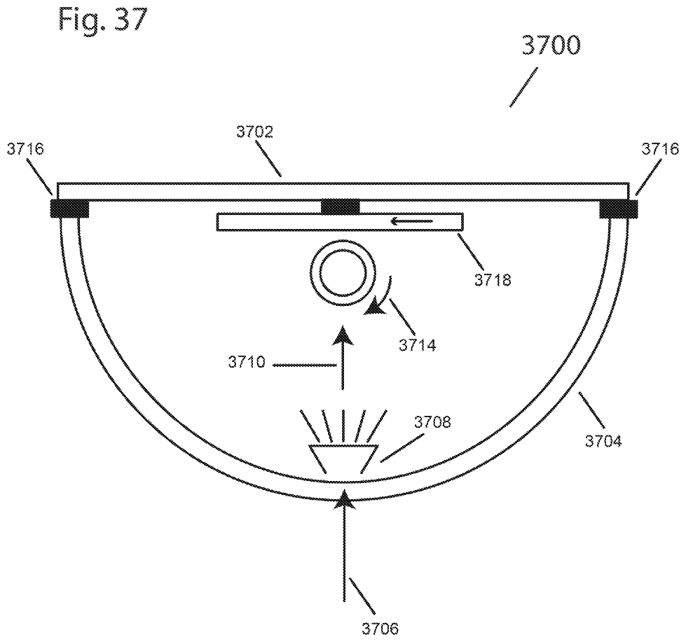

FIG. 37 shows a simplified cross section of a filter-making reactor 3700 and a cylindrical filter. The lid 3702 of a reactor 3700 supports an air seal 3716 and rotating working platform 3718 for filters 3712, sort of a motorized lazy-Susan. The lower shell 3704 of the reactor 3700 holds the gun 3708 which ionizes the hafnium wire 3706 that is slowly fed in and fogs the chamber's interior in a pseudo plasma state 3710 adhering to everything including a filter substrate where it cools. When plating 3710 filters onto a substrate, in this case a quartz cylinder 3712 the time and amount of hafnium or aluminum oxide 3706 fed determines the thickness of a particular layer. That thickness determines which wavelength(s) of light that layer can block or pass. By very slowly rotating 3714 the glass cylinder 3712 at a known rate around it long axis by mechanical means would be a further variable in the layer thickness equation. The rotation 3714 would allow a relatively even layer of hafnium 3710 all of the way around the cylinder 3712. The cylinder 3712 could also be the bulb 2902 where the outer surface of the bulb 2902 is plated with hafnium 3710 to become the filter 3712 as well. Approximately 100 layers are required for an ideal Far UV C filter. For simplicity the above explanation does not mention the voltages, temperatures, vacuum, gases, time, pressure, or monitoring equipment needed for properly operating a filter reactor, these elements are well known to those skilled in the art Only the pertinent, unique, and inventive elements have been discussed.

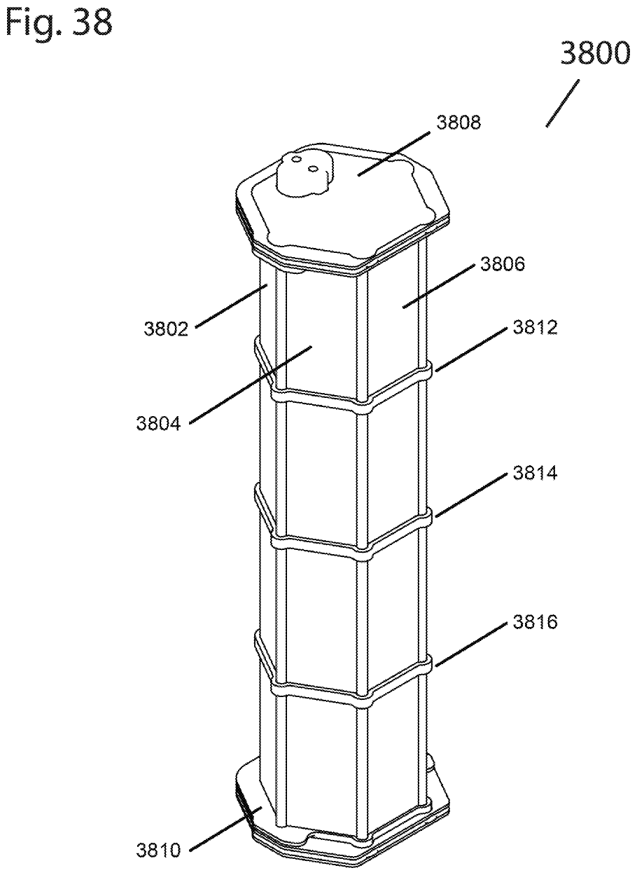

FIG. 38 Shows a UV C tower 3800 with multifaceted filters 3802, 3804, 3806 allowing for light to be generated in an omnidirectional fashion. The tower 3800 shown is a highly efficient and very powerful variation of the multifaceted filter invention. One segment is made of 4 or more filters 3802, 3804, 3806 (only 3 shown on a 6 filter fixture), 6 or 8 filters being ideal for a balance of manufacturability and efficiency, a segment splice 3812, and a bulb 800, 2300 positioned in the center of the filters 3802, 3804, 3806, such that the light coming from the bulb 800, 2300 strikes each filter 3802, 3804, 3806 at a zero-incident angle, which is perpendicular to each filter. A tower 3800 will also need a bottom cap 3810 and a top cap 3808 to completely enclose the light. More than one segment can be stacked for increased output and a more isotropic dispersion which will allow the light to wrap around 3 dimensional objects to a larger extent. When segments are stacked they use the segment splices 3812, 3814, 3816 which connect and seal the edges of the filters 3802, 3804, 3806. Rather than using short bulbs 800, 2300 and filters 3802, 3804, 3806 with multiple segments 3812, 3814, 3816 there could be advantages to using very long bulbs 800, 2300 and filters 3802, 3804, 3806. This would reduce the possibility of failure of so many components to just a few. The power supply would ideally be in the top or bottom cap as to not block the path of the bulb's 800, 2300 generated light. The bottom cap 3810 might include wheels or a robotic drive system for propulsion that are now becoming very common in industrial UV sanitizing systems.

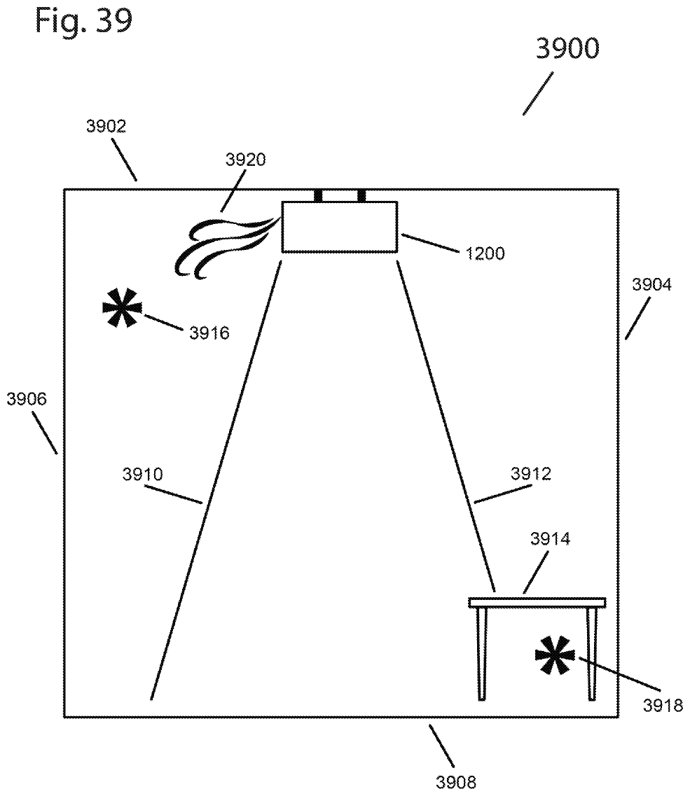

FIG. 39 shows a side view of a room 3900 being sanitized by the safe UV C fixture with a powerful fan. On the room's 3900 ceiling 3902 is mounted an inventive Far UV C fixture 1200 that has a powerful fan that blows currents of air 3920 to circulate the air in the room 3900. The room 3900 further consists of a left wall, a right wall, and a floor. The UV C fixture 1200 generates a beam of light that is defined on the left side 3910 and the right side 3912. The area between these to beam sides 3910, 3912 is the beam of light. Floating virus or bacteria 3916 that are not presently between the edges of the beam of light 3910, 3912 will shortly be moved into the beam 3910, 3912 by the current of air 3920 generated by the UV C fixture 1200. The beam might further be blocked by obstacles such as chairs or tables 3914 that could harbor virus or bacteria 3918. This harbored pathogen 3918 would be blown 3920 around the room by the inventive fixture 1200 such that it would eventually move through the beam 3910, 3912 of Far UV C light and be neutralized by the beam. Effectively cleaning the entire room without having to modify any HVAC systems or to add new HVAC systems. The high temperature components in the fixture 1200 might also kill pathogens 3916, 3918 as the fan blows air 3920 through the fixture 1200.

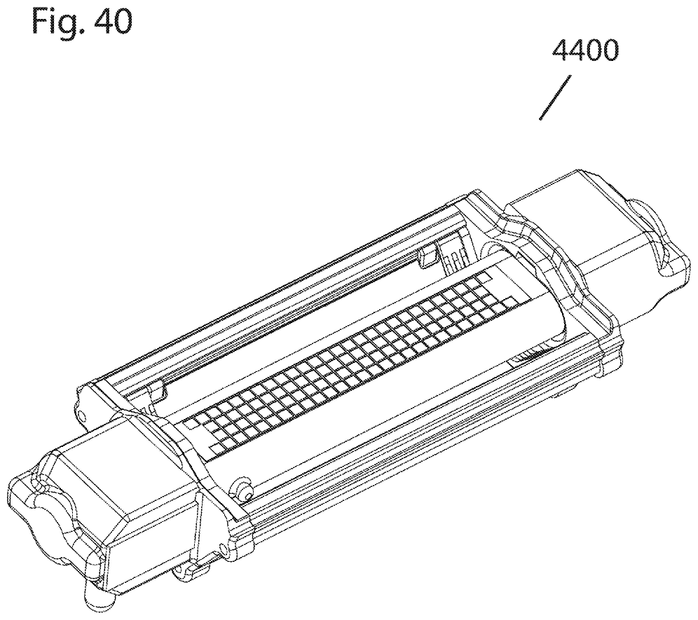

FIG. 40 shows a Isometric view of a simple cartridge bulb 4000. This bulb 4000 is considered simple because it does not have an integral filter like the more complex inventive bulb 900. The bulb cartridge 4000 might however have a quartz glass cover in a similar position as the filter of the complex bulb 900. This glass cover would allow the cartridge bulb 4000 to be sealed, and by being sealed it could contain a gas around the outside of the bulb. Such gas would be either nitrogen or a noble gas such that the gas would eliminate or radically minimize the production of ozone. This sealing would also eliminate dust and grime from getting on the reflectors and the inner quartz glass surfaces.

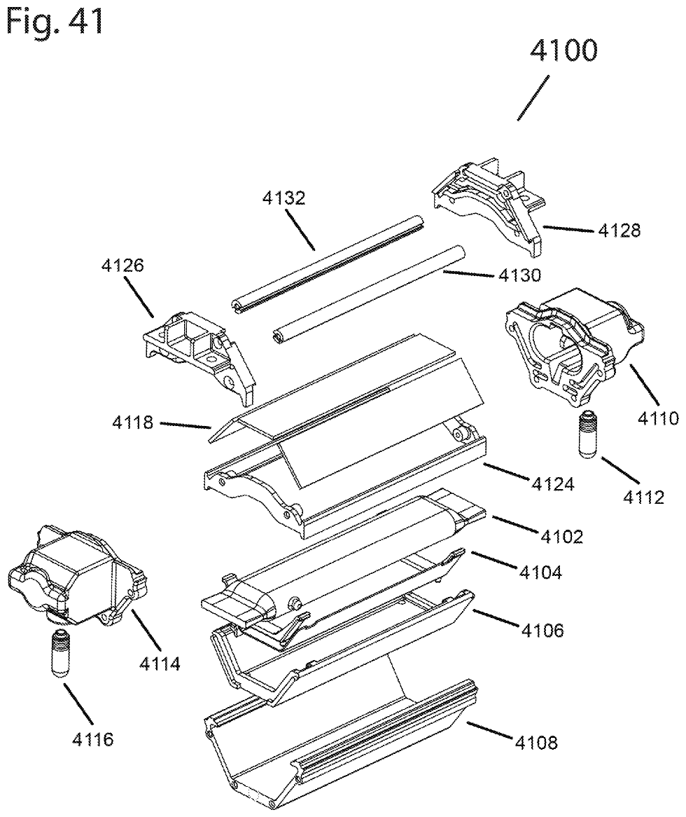

FIG. 41 Shows a more detailed explanation of the bulb cartridge's components 4100 as well as the construction of the ideal filter assembly using multifaceted filters. The bulb 4102 is situated above at least two reflectors 4104 that are held by the filter holder 4106. The filter holder is in direct contact with the heatsink which could be made of ceramic or metal and could have fins on the backside for greater thermal transfer to the air from a fixture's fan. The ends of the bulb are captured by ceramic end caps 4114, 4110. The last components of the bulb cartridge 4000 are the metal conductive pins 4116, 4112 and they provide a way for electrical power to be introduced to the bulb 4102.

The filter holder 4124 is designed to meet with the bulb cartridge 4000 in such a way as to block any light leakage between the two. The filter holder 4124 allows light to pass through its apertures while it holds 3 separate pass filters 4118. The filters 4118 butt against each other using either epoxy or slotted rods 4130, 4132 that block a tiny amount of light but do not allow any unfiltered light to pass, only filtered light passed the filters 4118. These rods 4130, 4132 also support and add rigidity to the filters 4118. These components 4124, 4118, 4130, 4132 are then held together using two end caps 4126, 4128 that stabilize the filter assembly and seal the ends from passing any unfiltered light. The multifaceted filter assembly is usually mounted on a fixture's front bezel.

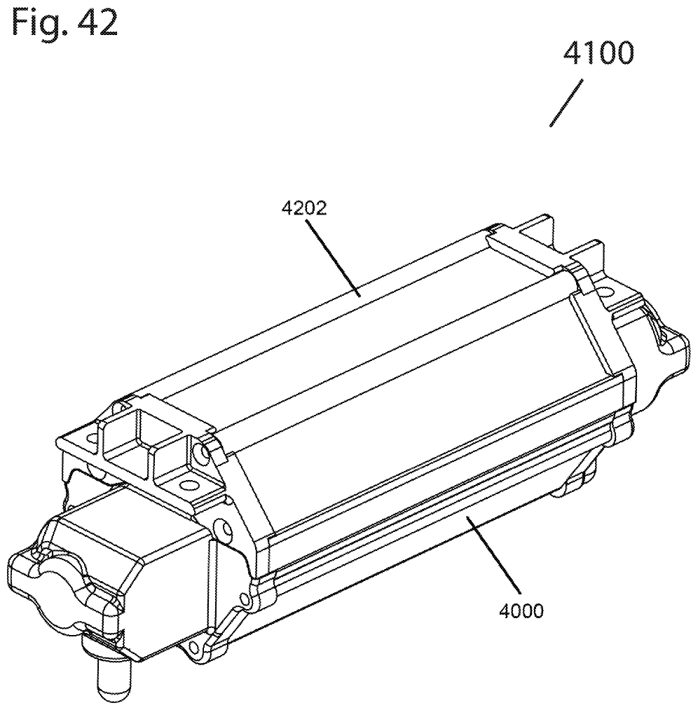

FIG. 42 Shows an isometric view 4100 of a simple cartridge bulb 4000 and filter assembly 4202. This further shows how well the bulb cartridge 4000 and filter assembly 4202 fit closely together.

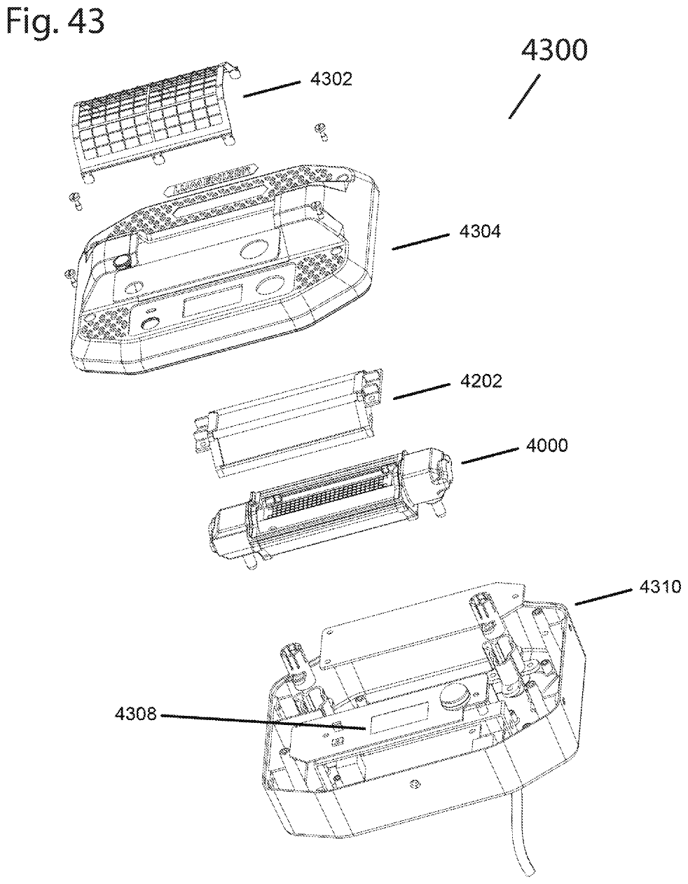

FIG. 43 Shows an exploded view of a single bulb fixture 4300 that uses multifaceted filters 4202. The base 4310 of the fixture 4300 holds the power supply 4308. The base 4308 also has receivers for the power pins and end caps of the bulb cartridge 4000. The bezel 4304 is a piece separate from the base 4308 and attached to the bezel are the multifaceted filter assembly and a possible safety cage. The safety cage is meant to keep people's fingers from touching the quartz and hafnium filters. Finger oil will block Far UV C and the filters might also get hot to a temperature high enough to burn fingers. The base 4308 and the bezel 4304 connect to make the bulk of the perimeter of the fixture 4300 and provide electrical and ultraviolet leakage protection when the unit operating. The two parts 4304, 4308 separate for replacing bulbs 4000, and of course there is a safety switch to detect the removal of the bezel 4304.

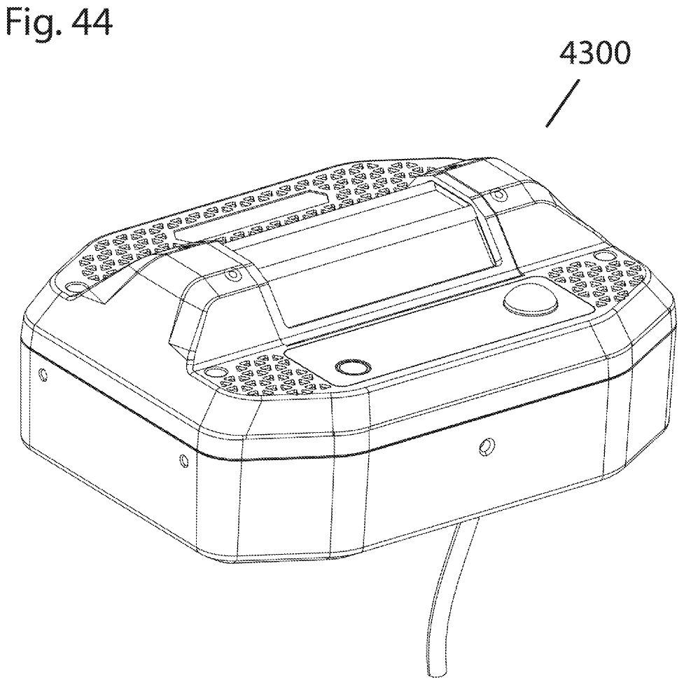

FIG. 44 Shows an isometric view of an assembled single bulb fixture 4300 that uses multifaceted filters 4202. This fixture could mounted flush or proud of a ceiling, it would be able to fit in ceiling cans designed for lighting purposes. A can based light fixture 4300 would ideally spread its light widely using multiple bulbs and filters aiming in multiple directions and or use light modifiers. It could be used in elevators, and other close spaces like bathrooms, hallways and such. It could also support different bezels to best fit with any given architectural style.

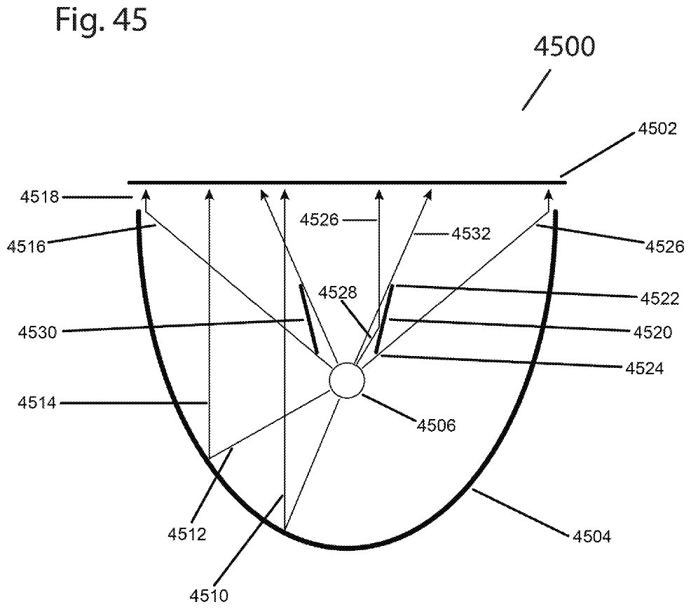

In FIG. 45 a cross section view of a directional high intensity filtered UV C light source 4500 is shown. This directional light engine 4500 at its core has a linear parabolic reflector 4504 with a capping pass filter 4502 covering the mirror's 4504 aperture, with a Far UV C bulb 4506 positioned at the locus or focus point of the parabolic reflector 4504. The bulb 4506 shown is round but any Far UV C bulb 4506 geometry should work if placed at the locus. A light ray 4508 that comes from the bulb 4506 and goes backward will strike the reflector 4504 and the ray takes a new path 4510 that is very close to perpendicular to the filter 4502. A light ray 4512 that goes out sideways and strikes the reflector 4504 will also have a path 4514 that will hit the filter 4502 at a near perpendicular angle. Even a light ray 4516 that goes out slightly forward will also strike the mirror 4504 and be redirected to a path 4518 that is perpendicular to the filter 4502. There is light 4528 going forward to the filter 4502 that doesn't bounce of a reflector 4504 or mirror 4504 and if light 4532 is within about 20 degrees of perpendicular most will pass through the filter 4502. Light rays that go forward and don't bounce off of the parabolic reflector 4504 but are going to hit the filter 4502 at an angle greater than 20 degrees from perpendicular should be intercepted by a small linear reflector 4520 that is angled such that the light ray 4528 is redirected in a direction 4526 which is perpendicular to the filter 4502, effectively harvesting light that would have been absorbed and lost to the filter 4502, now being directed at an angle that will allow it to pass through the filter 4502 adding to the system efficiency. The inside end 4524 of this small linear reflector would be positioned to reflect light that would miss just the reflector 4504 and the outside end 4522 of the small linear reflector would be positioned where light paths would be greater than 20 degrees from perpendicular to the filter 4502. This highly efficient method would be repeated on the opposite side of the reflector 4504 using a second small linear reflector 4530.

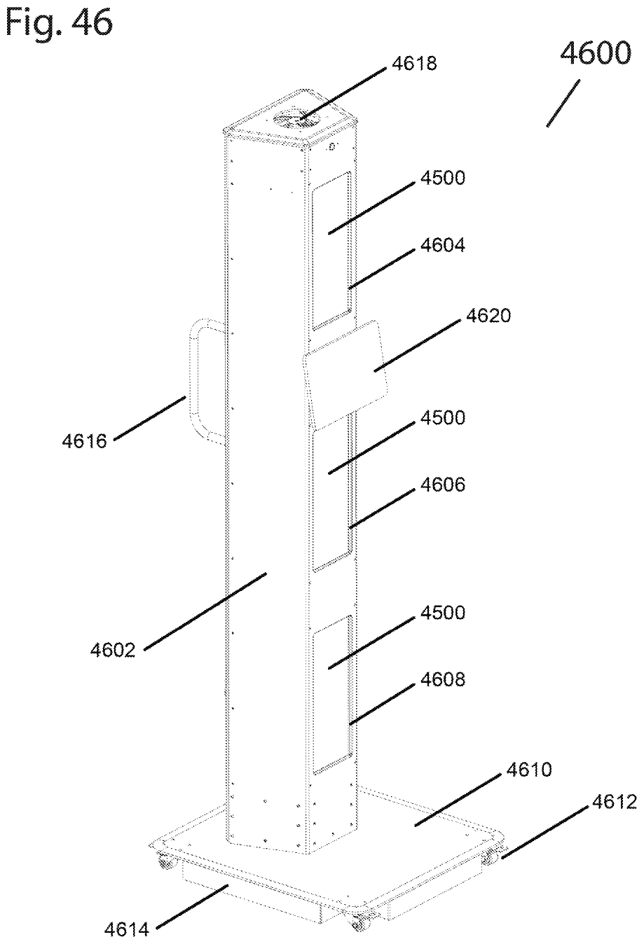

FIG. 46 shows an isometric view of a directional UV C tower 4600. The tower 4600 as shown uses 3 of the inventive directional high intensity filtered UV C light sources 4500. The 3 directional light engines 4500 shine through 3 apertures 4604, 4606, 4608, all in the same direction. The 3 apertures 4604, 4606, 4608 are shown as an example but any number could be used depending on the need of the user. The tower 4600 would have a chassis 4602 or body 4602, a base 4610 that would ideally have wheels 4612 and ideally hold all power supplies 4614. The tower 4600 could have a visual interface 4620 such as an iPad 4620 or LCD display 4620 and provide an IoT interface. The display 4620 could show time or instructions to users for proper disinfection practices as well as take inputs from users. The tower 4600 could include sensors 4622 or cameras 4622 that detect the body temperature and health of the users. Ideally the tower 4600 would have a fan 4618 for cooling and a handle 4616 of some type to ease the process of moving the tower 4600.

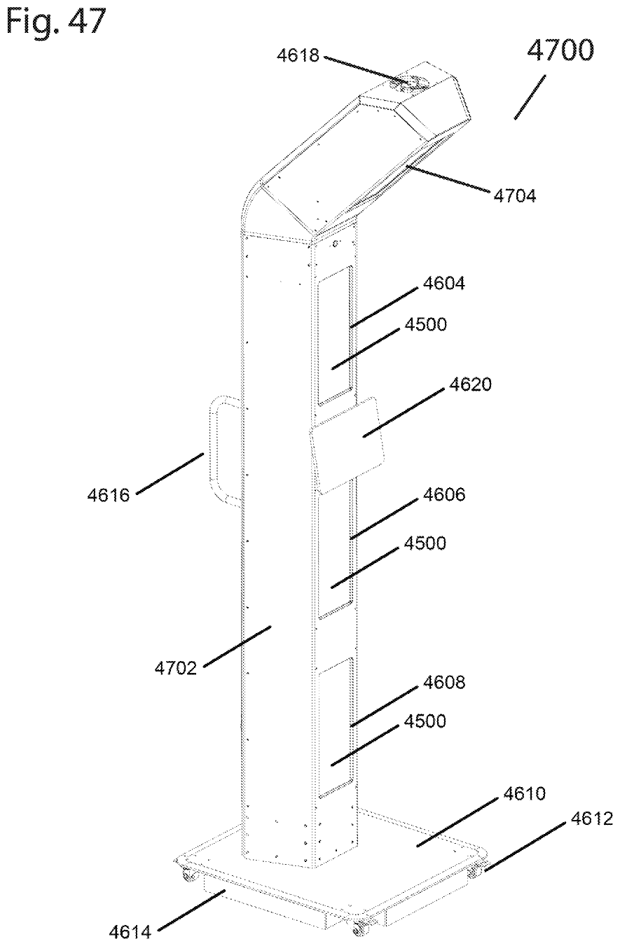

FIG. 47 Shows an isometric view of a directional UV C gateway tower 4700. The tower 4700 as shown uses 4 of the inventive directional high intensity filtered UV C light sources 4500. The 4 directional light engines 4500 shine through 4 apertures 4604, 4606, 4608, 4704, 3 of these apertures 4604, 4606, 4608 in the same direction and one 4704 pointed down The 4 apertures 4604, 4606, 4608, 4704 are shown as an example but any number could be used depending on the need of the user. The gateway tower 4700 similar to the straight tower 4600 would have a chassis 4602 or body 4602, a base 4610 that would ideally have wheels 4612 and ideally hold all power supplies 4614. The tower 4700 could have a visual interface 4620 such as an iPad 4620 or LCD display 4620. The display 4620 could show time or instructions to users for proper disinfection practices as well as take inputs from users and provide an IoT interface. The tower 4700 could include sensors 4622 or cameras 4622 that detect the body temperature and health of the users. Ideally the tower 4700 would have a fan 4618 for cooling and a handle 4616 of some type to ease the process of moving the tower 4700.

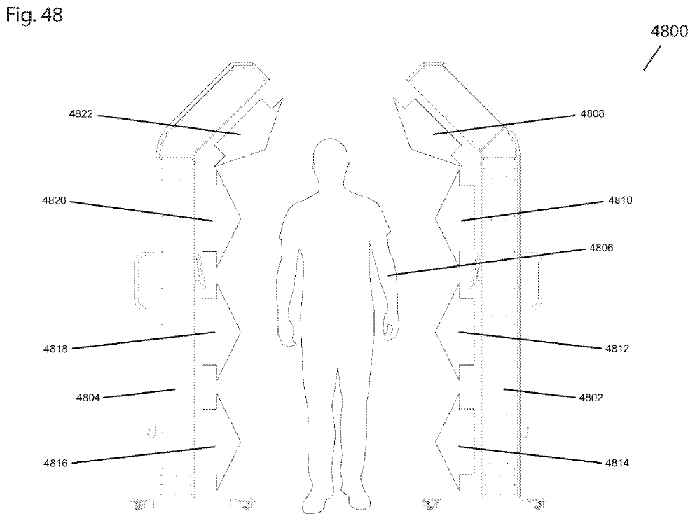

FIG. 48 shows a side view of two directional towers 4700, 4802, 4804 forming a UV C gateway 4800. A person 4806 would walk or pass between the right tower 4802 and the left tower 4804. Light 4808, 4810, 4812, 4814, 4816, 4818, 4820, 4822 would be directed in a highly concentrated manner so that the person 4806 would need an exposure of a few seconds to be surface disinfected. Light would come down 4808, 4822 from above the head as well as from the sides 4810, 4812, 4814, 4816, 4818, 4820, to cover all areas as the person 4806 passes through.

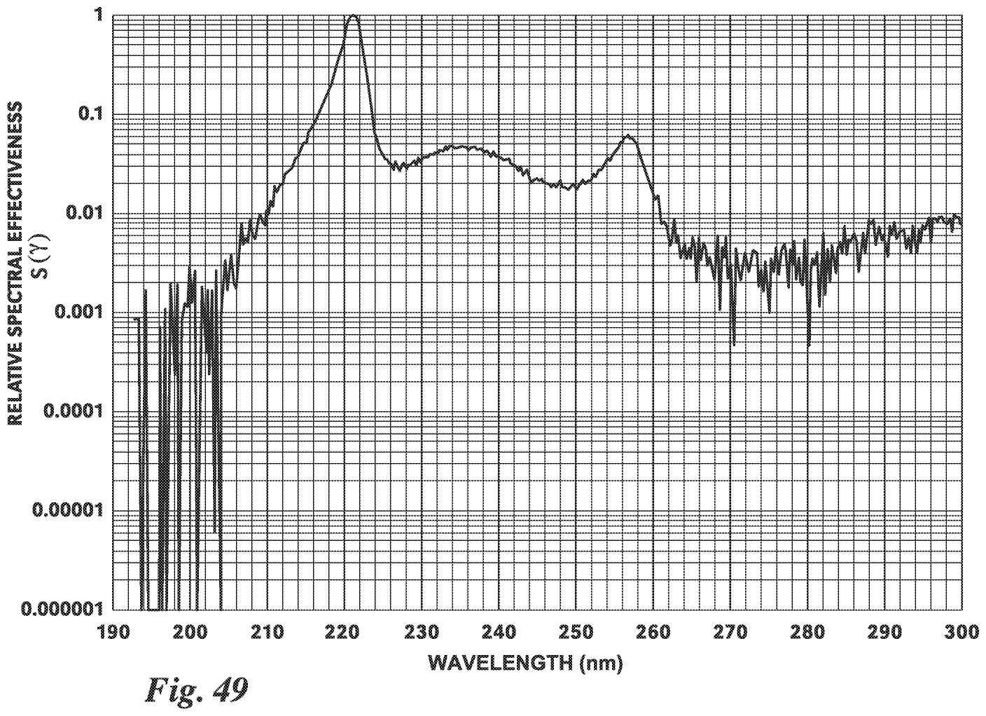

FIG. 49 shows logarithmic graph 4900 of an unfiltered 222 nm bulb. This graph 4900 is very similar to FIG. 5 except for the logarithmic function that shows more detail at the lower intensity levels. Following the 222 nm bulb spectrum 4902 we can see the portions that are divided by the 1% of peak brightness line 4904. The areas under the line do not need to be filtered. The areas over the line that are longer than 240 nm 4906 need to be filtered. The 222 nm bulb spectrum 4902 drops below the 1% of peak brightness line 4904 at 262 nm 4908 and stays below until 288 nm 4910 where the 222 nm bulb spectrum 4902 approaches almost the 1% of peak brightness line until 315 nm 4912 where the wavelengths that are longer are considered safe. We now know that we need to start filtration 4 nm shorter from any bad wavelengths and 240 nm 4906 requires us the begin filtering at 236 nm or shorter. That filtration continues with all longer wavelengths until 262 nm 4908. We also start filtering at 288 nm 4910 rather than at 284 nm because the intensity of the 222 nm bulb spectrum 4902 is slightly short of the 1% line and there is at least partial filtering directly at 288 nm 4910 and that filtering is continuous to 315 nm 4912.

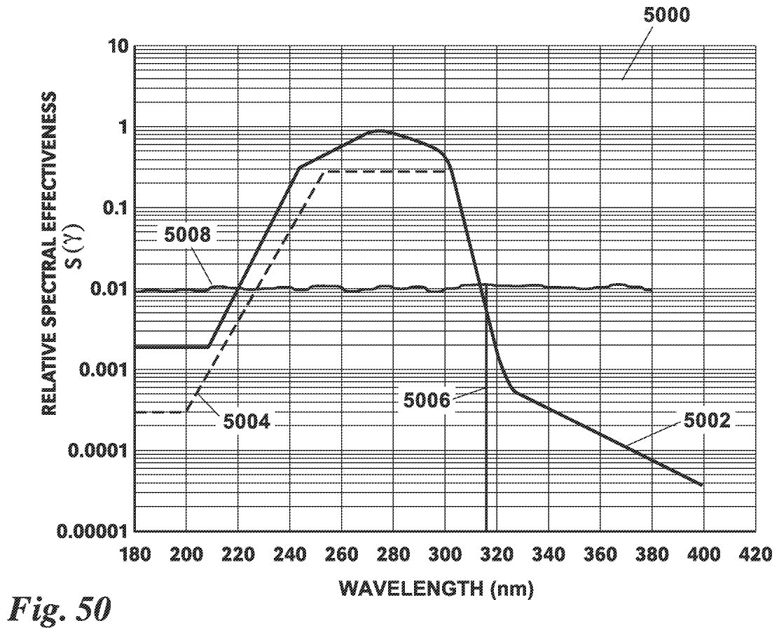

FIG. 50 shows a graph of relative spectral effectiveness 5000 or hazard function of UV on human tissue. This graph came from the International Electrotechnical Commission (IEC) who set standards of safety regarding exposure to UV C. Note that there are two different curves, the first curve is of eye exposure 5002 and the second is skin exposure 5004. If you look at the crossing point of 315 nm line 5006 with the combined eye/skin 5002, 5004 line, they cross at the 1% of peak line 5008. This is why the IEC set 315 nm 5006 as the cutoff point for dangerous wavelengths, the combined eye/skin 5002, 5004 line drops below 1% of peak 5008 there. Scientific testing has shown that the left side of the graph is not entirely correct and is in the process of being updated by the IEC. That is why it is safely possible to cutoff at 239 nm/240 nm and expose humans to 200 nm to 237 nm wavelengths. Further research has shown that the wavelengths between 220 nm and 230 nm are the real virus killing wavelengths, so it is important to extend the filtration as far as possible to the longer wavelengths to allow as much of the 230 nm to be passed through the filter. That combined with the knowledge of the 4 nm offset is why going to cutoff wavelengths longer than 234 nm are critical for efficiency. 234 nm, 235 nm, and 236 nm are the most efficient safe wavelengths to use as a starting point.

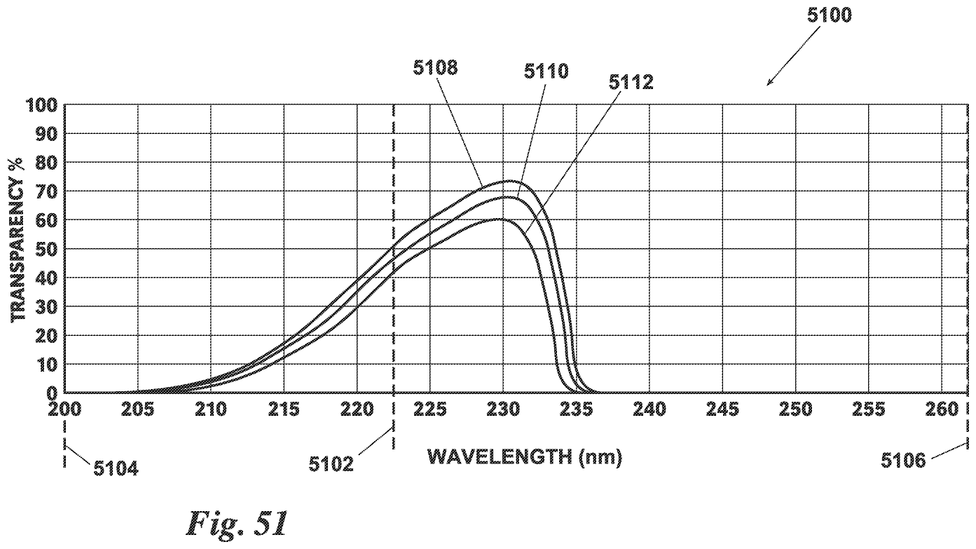

FIG. 51 shows a graph of a filter's pass curves 5100. The key bulb wavelength of 222 nm is shown by the vertical 222 nm line 5102, and the shortest wavelength 200 nm 5104, and the upper end 262 nm 5106 of this drawing's spectrum. The 3 traces 5108, 5110, 5112 shown on the graph 5100 are of 234 nm cutoff filter 5112, 235 nm cutoff filter 5110, and a 236 nm cutoff filter 5108. This is not the light coming through the filter, this what light is allowed through the filter. This is for comparing the different wavelengths and their effectiveness and efficiency. The 234 nm cutoff filter 5112 crosses the vertical 222 nm line 5102 at the 40% transparency line. The 235 nm cutoff filter 5110 crosses the vertical 222 nm line 5102 at 46% transparency. The 236 nm cutoff filter 5108 crosses the vertical 222 nm line 5102 at 52% efficiency. There is substantial efficiency improvement shown to result in the conclusion that a 236 nm cutoff filter 5108 is by far the best safe solution.

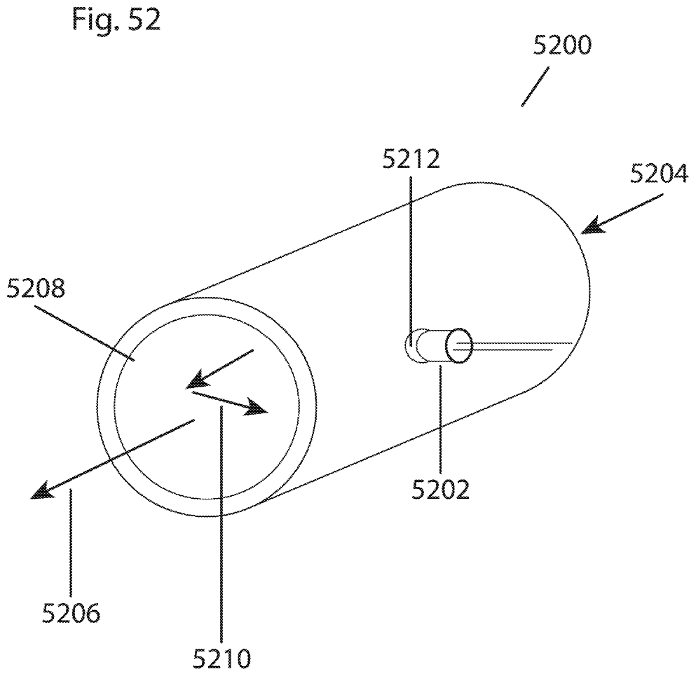

FIG. 52 Shows an isometric view of a reflective air exhaust chamber assembly 5200 using a 265 nm LED 5202 to reduce ozone 5204 passing through the chamber 5200. The interior wall 5208 of the chamber 5200 is made of UV C reflective material allowing multiple bounces 5210 of the light emitted by the small and inefficient 265 nm LED 5202. These bounces 5210 multiply and intensify the effect of the low energy 265 nm LED 5202 and yet not allowing significant amounts of the light 5202 to spill out of the chamber 5200. 265 nm is one of the ideal wavelengths that removes ozone 5204 so that air expelled 5206 from the chamber 5200 is ozone reduced or ozone free. The inventive chamber 5200 would be situated in the exhaust air path of a FAR UV C fixture 1200, as a generic example of chamber used with a FAR UVC fixture but does not limit the type of FAR UV C fixture described by the present invention.

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are encompassed within the scope and spirit of this invention.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A UV light source having an easily removed and replaced reflective light modifier.

2. The UV light source of claim 1 wherein the light modifier is at least one reflector

3. The UV light source of claim 2 wherein the least one reflector is curved.

4. The UV light source of claim 2 wherein the least one reflector is perforated.

5. The UV light source of claim 1 wherein the light modifier forms a narrow pattern.

6. The UV light source of claim 1 wherein the light modifier forms a circular pattern.

7. The UV light source of claim 1 wherein the light modifier forms a flat pattern.

8. The UV light source of claim 1 wherein the light modifier forms an asymmetrical pattern.

9. The UV light source of claim 1 wherein the light modifier forms an upper air pattern.

10. The UV light source of claim 1 wherein the UV light source is a FAR UV C light source.

10. The UV light source of claim 1 wherein the UV light source is a FAR UV C excimer bulb.

11. The UV light source of claim 1 wherein the UV light source is a UV C LED.

12. The UV light source of claim 1 wherein the UV light source is a 405 nm LED.
